(19) United States
(12) Patent Application Publication
(10) Pub. No.: US 2011/0223594 A1
(43) Pub. Date: Sep. 15, 2011

(54) METHODS, KITS AND COMPOSITIONS FOR DETERMINING SEVERITY AND SURVIVAL OF HEART FAILURE IN A SUBJECT

(75) Inventors: Peter Vanburen, Burlington, VT (US); Jun Ma, Toronto (CA); Choong-Chin Liew, Toronto (CA)

(73) Assignee: GENENEWS INC., Richmond Hill, ON (CA)

(21) Appl. No.: 13/002,007

(22) PCT Filed: Jun. 29, 2009
(86) PCT No.: PCT/CA2009/000900

§ 371 Date: May 26, 2011

Related U.S. Application Data
(60) Provisional application No. 61/076,901, filed on Jun. 30, 2008.

(51) Int. Cl. C12Q 1/68; C07H 21/00; G06F 19/00

(52) U.S. Cl. 435/6.11; 435/6.1; 435/6.12; 536/24.33; 702/19

(57) ABSTRACT

The application provides a method of determining a severity of heart failure in a human test subject, by determining a level of RNA encoded by one or more heart failure marker genes in blood of the test subject compared to controls. The application also provides a method of determining survival outcome and allows the ranking of test subjects based on the level of RNA encoded by one or more survival associated genes.

### FIELD OF THE DISCLOSURE

The disclosure relates to methods, kits and compositions for determining the severity and survival outcome of a subject with heart failure. More particularly, the disclosure relates to methods, kits and compositions for determining the severity and survival outcome by measuring a level of one or more gene products in blood of the subject.

### BACKGROUND OF THE DISCLOSURE

Heart failure is increasing as a public health concern and rapidly growing as an economic burden. The enormous public health and economic burdens imposed by heart failure can be decreased only by introducing improved therapies and better patient management. The genomic approaches to disease that have revolutionized biologic and biomedical research over the past 10 years hold significant promise in tackling these issues.

Heart failure results from structural or functional cardiac disorders that lead to insufficient supply of blood throughout the body. With an aging population, heart failure has become a major public health concern with its incidence continuing to increase: the condition currently affects more than five million people in the United States, and more than 500,000 new cases occur annually (Rosamond et al., 2007). While advances in the management of heart failure have modestly improved outcomes in patients with this disease, heart failure still remains the leading hospital admission diagnosis in elderly patients and carries a 5 year mortality of nearly 50% (Roger et al., 2004; Schocken et al., 2008). Thus the overall morbidity and mortality of this disease remain unacceptably high. As such better diagnostic strategies are required to aid in defining the prognosis and treatment of patients with heart failure.

Heart failure has long been recognized as a systemic disease directly affecting circulating level of numerous neurohormones, cytokines and inflammatory markers (Braunwald, 2008; Mann and Bristow, 2005). These circulating factors directly affect (largely adversely) intracellular signaling and consequent gene expression which has been well demonstrated in the myocardium. Specifically, significant elevation of gene expression associated with cell growth, signal transduction and cell defense have been demonstrated using gene expression profiling with microarray (Cunha-Neto et al., 2005; Kittleson et al., 2005). Thus myocardial gene expression likely reflects direct tissue changes associated with the cardiomyopathic process as well as consequential alterations in gene expression secondary to the humoral response of the disease state.

Given the systemic nature of heart failure and the protean nature of neurohormonal signaling, other tissues are also affected by heart failure state. Expression profiling of blood samples has been successfully applied to identify blood expression patterns associated with coronary artery disease (Ma and Liew, 2003) and with plasma lipid levels (Ma et al., 2007).

There remains a need for blood expression signatures as diagnostic and prognostic tools in heart failure management.

### SUMMARY OF THE DISCLOSURE

The present inventors have shown novel blood markers for determining the severity and survival outcome of heart failure in a subject. This use can be effected in a variety of ways as further described and exemplified herein.

Accordingly, in one aspect there is provided a method of determining a severity of heart failure in a human test subject, the method comprising, for each gene of a set of one or more genes listed in Table 2: a) providing test data representing a level of RNA encoded by the gene in blood of the test subject; b) providing positive control data representing levels of RNA encoded by the gene in blood of human control subjects having a categorized severity of heart failure; and c) comparing the level of step a) to the levels in blood of control subjects to thereby determine a value indicating whether the test data corresponds to the positive control data; wherein a correspondence between the test data and the positive control data indicates that the test subject has the categorized severity of heart failure. In one embodiment, the set of genes comprises or consists of an ASGR2 gene and a STAB1 gene. In another embodiment, the control data comprises the average level in control subjects. The categorized severity may be compensated heart failure or decompensated heart failure. The method may further comprise determining a level of RNA encoded by the gene in blood of the test subject, thereby providing the test data. The method may further comprise determining levels of RNA encoded by the gene in blood of human subjects having the categorized severity of heart failure, thereby providing the positive control data. Step c) may be effected by: inputting, to a computer, the test data, wherein the computer is for comparing data representing a level of RNA encoded by the gene in blood of a human subject to levels of RNA encoded by the gene in subjects having the categorized severity of heart failure, to thereby output a value indicating whether the test data corresponds to the positive control data; and causing the computer to compare the test data to the positive control data, to thereby output the value indicating whether the test data corresponds to the positive control data.

In another aspect there is provided a method of determining a severity of heart failure in a human test subject, the method comprising, for each gene of a set of one or more of the genes listed in Table 2 (a) determining a level of RNA encoded by the gene in blood of the test subject, thereby generating test data; (b) providing a first positive control data representing levels of RNA encoded by the gene in blood of human control subjects having a first categorized severity of heart failure; and (c) comparing the levels of a) and b); wherein a correspondence between the test data and the positive control data indicates that the test subject has the first categorized severity of heart failure. In one embodiment, the one or more genes is ASGR2, C3AR1 and/or STAB1. The first categorized severity may be compensated heart failure or decompensated heart failure.

In a further aspect, the method further comprises providing a second positive control data representing levels of RNA encoded by the gene in blood of human control subjects having a second categorized severity of heart failure, wherein correspondence between the test data and the second positive control data indicates that the test subject has the second categorized severity of heart failure. In one embodiment, the first categorized severity is compensated heart failure and the second categorized severity is decompensated heart failure.

According to further features described below the determining of the level of RNA encoded by the gene in blood of the test subject is determined as a ratio to a level of RNA encoded by the gene in blood of a healthy test subject.

In another aspect, the method further comprises determining levels of RNA encoded by the gene in blood of a population of human subjects having the first categorized severity of heart failure, thereby providing the positive control data representing the levels of RNA encoded by the gene in blood of human control subjects having the first categorized severity of heart failure. In yet another aspect, the method further comprises determining levels of RNA encoded by the gene in blood of a population of human subjects having the second categorized severity of heart failure, thereby providing the positive control data representing the levels of RNA encoded by the gene in blood of human control subjects having the second categorized severity of heart failure.

In a further aspect the method further comprises providing a third control data representing levels of RNA encoded by the gene in blood of human control subjects which are healthy, and wherein step c) is effected by comparing the test data to the first or second positive control data and the third control data, wherein correspondence between the test data and the first or second positive control data and not the third control data indicates that the test subject has the first or second categorized severity of heart failure.

According to another aspect, there is provided a computer-based method of determining a severity of heart failure in a human test subject, the method comprising inputting, to a computer, test data representing a level of RNA encoded by one or more of the genes listed in Table 2 in blood of the test subject; and causing the computer to compare the test data to a first positive control data representing levels of RNA encoded by the gene in blood of human control subjects having a first categorized severity of heart failure, wherein correspondence between the test data and the first positive control data indicates that the test subject has the first categorized severity of heart failure. In one embodiment, the one or more genes is ASGR2, C3AR1 and/or STAB1.

In a further aspect, there is provided a method of monitoring the progression of heart failure in a human subject, the method comprising for each gene of a set of one or more of ASGR2, C3AR1, STAB1, KRCC1, KYNU, LOH11CR2A, TMEM144, FKBP1B, VCAN, LTA4H, MGST1, or NOTCH2NL (a) determining a level of RNA encoded by an ASGR2 gene in blood of the subject at a first time point; (b) determining a level of RNA encoded by an ASGR2 gene in blood of the subject at a second time point; (c) comparing the levels in a) and b); wherein an increased level at the second time point indicates a progression of heart failure. In one embodiment, the one or more genes is/are ASGR2, C3AR1 and/or STAB1.

In another aspect, there is provided a method of monitoring the progression of heart failure in a human subject, the method comprising, for each gene of a set of one or more of FAM84B, RBL2, XRCC5, TNRC6C, ZBTB44, HERC1, SNORA72, WIPF1, PPP1R2, C4orf30, KIAA0888, TMEM106B, NR3c2, KLHL24, FLJ31306, MAP2K6, SATB1, WHDC1L1, EDG1, MBIP, RSU1, DYRK2, DOCK9, LOC283666, SLC4A7, ODF2L, PAQR8, or C14orf64 or the genes listed in Table 4 (a) determining a level of RNA encoded by the gene in blood of the subject at a first time point; (b) determining a level of RNA encoded by the gene in blood of the subject at a second time point; (c) comparing the levels in a) and b); wherein an decreased level at the second time point indicates a progression of heart failure.

According to yet another aspect, there is provided a computer-based method of monitoring the progression of heart failure in a human subject, the method comprising inputting, to a computer, test data representing a level of RNA encoded by one or more of ASGR2, C3AR1, STAB1, KRCC1, KYNU, LOH11CR2A, TMEM144, FKBP1B, VCAN, LTA4H, MGST1, or NOTCH2NL gene in blood of the subject at a first and second time point; and causing the computer to compare the data of the first time point to the data of the second time point, and to determine whether the level of RNA encoded by the gene in blood of the subject is increased at the second time point compared to the level of RNA encoded by the gene in blood of the subject at the first time point, wherein a determination that the level of RNA encoded by the gene in blood of the test subject is increased at the second time point indicates the progression of heart failure. In one embodiment, the one or more genes is ASGR2, C3AR1 and/or STAB1.

In an additional aspect, there is provided a computer-based method of monitoring the progression of heart failure in a human subject, the method comprising inputting, to a computer, test data representing a level of RNA encoded by one or more of FAM84B, RBL2, XRCC5, TNRC6C, ZBTB44, HERC1, SNORA72, WIPF1, PPP1R2, C4orf30, KIAA0888, TMEM106B, NR3c2, KLHL24, FLJ31306, MAP2K6, SATB1, WHDC1L1, EDG1, MBIP, RSU1, DYRK2, DOCK9, LOC283666, SLC4A7, ODF2L, PAQR8, or C14orf64 gene or the genes listed in Table 4 in blood of the subject at a first and second time point; and causing the computer to compare the data of the first time point to the data of the second time point, and to determine whether the level of RNA encoded by the gene in blood of the subject is decreased at the second time point compared to the level of RNA encoded by the gene in blood of the subject at the first time point, wherein a determination that the level of RNA encoded by the gene in blood of the test subject is decreased at the second time point indicates the progression of heart failure.

In another aspect, there is provided a method of classifying a human test subject as having decompensated heart failure, the method comprising (a) determining a level of RNA encoded by each gene of a set of one or more of ASGR2, C3AR1, STAB1, KRCC1, KYNU, LOH11CR2A, TMEM144, FKBP1B, VCAN, LTA4H, MGST1, or NOTCH2NL in blood of the test subject, thereby generating test data; (b) providing control data representing a level of RNA encoded by the gene in blood of human control subjects not having decompensated heart failure; and (c) comparing the test data to the control data, wherein a determination in step (c) that the level of RNA encoded by the gene in blood of the test subject is higher than the level of RNA encoded by the gene in blood of human control subjects not having decompensated heart failure is used to classify the test subject as having decompensated heart failure.

In yet another aspect, there is provided a method of classifying a human test subject as having decompensated heart failure, the method comprising for each gene of a set of one or more of FAM84B, RBL2, XRCC5, TNRC6C, ZBTB44, HERC1, SNORA72, WIPF1, PPP1R2, C4orf30, KIAA0888, TMEM106B, NR3c2, KLHL24, FLJ31306, MAP2K6, SATB1, WHDC1L1, EDG1, MBIP, RSU1, DYRK2, DOCK9, LOC283666, SLC4A7, ODF2L, PAQR8, or C14orf64 (a) determining a level of RNA encoded by the gene in blood of the test subject, thereby generating test data; (b) providing control data representing a level of RNA encoded by the gene in blood of human control subjects not having decompensated heart failure; and (c) comparing the test data to the control data, wherein a determination in step (c) that the level of RNA encoded by the gene in blood of the test subject is lower than the level of RNA encoded by the gene in blood of human control subjects not having decompensated heart failure is used to classify the test subject as having decompensated heart failure.

In a further aspect there is provided a computer-based method of classifying a human test subject as having decompensated heart failure, the method comprising for each gene of a set of one or more of ASGR2, C3AR1, STAB1, KRCC1, KYNU, LOH11CR2A, TMEM144, FKBP1B, VCAN, LTA4H, MGST1, or NOTCH2NL inputting, to a computer, test data representing a level of RNA encoded by the gene in blood of the test subject; and causing the computer to compare the test data to control data representing a level of RNA encoded by the gene in blood of human control subjects not having decompensated heart failure, and to determine whether the level of RNA encoded by the gene in blood of the test subject is higher than the level of RNA encoded by the gene in blood of human control subjects not having decompensated heart failure, wherein a determination that the level of RNA encoded by the gene in blood of the test subject is higher than the level of RNA encoded by the gene in blood of human control subjects not having decompensated heart failure is used to classify the test subject as having decompensated heart failure.

In yet a further aspect, there is provided a computer-based method of classifying a human test subject as having decompensated heart failure, the method comprising for each gene of a set of one or more of FAM84B, RBL2, XRCC5, TNRC6C, ZBTB44, HERC1, SNORA72, WIPF1, PPP1R2, C4orf30, KIAA0888, TMEM106B, NR3c2, KLHL24, FLJ31306, MAP2K6, SATB1, WHDC1L1, EDG1, MBIP, RSU1, DYRK2, DOCK9, LOC283666, SLC4A7, ODF2L, PAQR8, or C14orf64 inputting, to a computer, test data representing a level of RNA encoded by the gene in blood of the test subject; and causing the computer to compare the test data to control data representing a level of RNA encoded by the gene in blood of human control subjects not having decompensated heart failure, and to determine whether the level of RNA encoded by the gene in blood of the test subject is lower than the level of RNA encoded by the gene in blood of human control subjects not having decompensated heart failure, wherein a determination that the level of RNA encoded by the gene in blood of the test subject is lower than the level of RNA encoded by the gene in blood of human control subjects not having decompensated heart failure is used to classify the test subject as having decompensated heart failure.

In another aspect, there is provided a method of determining whether a human subject with heart failure has a prognosis of mortality, the method comprising for each gene of a set of one or more of the genes set forth in Table 3 (a) determining a level of RNA encoded by the gene in blood of the test subject, thereby generating test data; (b) providing positive control data representing levels of RNA encoded by the gene in blood of human control subjects having a prognosis of mortality; and (c) comparing the levels of a) to b), wherein a correspondence between the test data and the positive control data indicates that the test subject has a prognosis of mortality. In one embodiment, the determining whether the test data corresponds to the positive control data is effected by applying to the test data a mathematical model derived from the positive control data, and wherein the mathematical model is for determining the whether a level of RNA encoded by the gene corresponds to the positive control data. In one embodiment, the set of one or more genes comprise FAM134B, MGAT4A, ZCCHC14 or CD28.

In yet another aspect, there is provided a computer-based method for determining whether a human subject with heart failure has a prognosis of mortality, the method comprising inputting, to a computer, test data representing a level of RNA encoded by one or more of the genes set forth in Table 3 in blood of the test subject; and causing the computer to compare the test data to a positive control data representing levels of RNA encoded by the gene in blood of human control subjects having a prognosis of mortality, wherein correspondence between the test data and the positive control data indicates that the test subject has the prognosis of mortality.

According to another aspect, there is provided a method of ranking two or more test subjects having heart failure according to risk of death, the method comprising for each gene of a set of one or more of the genes set forth in Table 3: (a) determining a level of RNA encoded by the gene in blood of each test subject, thereby generating test data; (b) calculating the risk score for each test subject based on the level of expression in (a); (c) ranking the risk scores of the test subjects, wherein the test subjects are ranked according to risk of death.

In yet a further aspect, there is provided a computer-based method of ranking two or more test subjects having heart failure according to risk of death, the method comprising for each gene of a set of one or more of FAM134B, MGAT4A, ZCCHC14 or CD28: inputting, to a computer, test data representing a level of RNA encoded by one or more of a FAM134B, MGAT4A, ZCCHC14 or CD28 gene in blood of each test subject; causing the computer to apply the test data to a relative risk equation; and causing the computer to rank the results of each test subject, wherein the computer provides a ranking of the test subjects based on the relative risk.

According to still another aspect of the invention there is provided a kit comprising packaging and containing, for each gene of a set of one or more of the genes listed in Table 2, a primer set capable of generating an amplification product of DNA complementary to RNA encoded, in a human subject, only by the gene. In one embodiment, the set of one or more genes comprises ASGR2, C3AR1 and/or STAB1. In another embodiment, the set of one or more genes comprises or consists of an ASGR2 gene and a STAB1 gene.

According to further features of the invention described below, the kit further comprises a computer-readable medium having instructions stored thereon that are operable when executed by a computer for comparing test data representing a level of RNA encoded by the gene in blood of a human test subject to a first positive control data representing levels of RNA encoded by the gene in blood of human control subjects having a first categorized severity of heart failure, to thereby output data representing a value indicating whether the test data and the positive control data correspond to each other, wherein correspondence between the test data and the first positive control data indicates that the test subject has the first categorized severity of heart failure.

In another embodiment, the computer readable medium further has instructions stored thereon that are operable when executed by a computer for comparing a second positive control data representing levels of RNA encoded by the gene in blood of human control subjects having a second categorized severity of heart failure, wherein correspondence between the test data and the second positive control data indicates that the test subject has the second categorized severity of heart failure.

In yet another aspect, there is provided a kit comprising packaging and containing, for each gene of a set of one or more of the genes set forth in Table 3, a primer set capable of generating an amplification product of DNA complementary to RNA encoded, in a human subject, only by the gene. In one embodiment, the one or more genes comprises FAM134B, MGAT4A, ZCCHC14 and/or CD28.

According to further features of the invention described below, the kit further comprises a thermostable polymerase, a reverse transcriptase, deoxynucleotide triphosphates, nucleotide triphosphates and/or enzyme buffer.

According to further features of the invention described below, the kit further comprises at least one labeled probe capable of selectively hybridizing to either a sense or an antisense strand of the amplification product.

According to further features of the invention described below, the level of RNA encoded by the gene in blood of the test subject is determined via quantitative reverse transcriptase-polymerase chain reaction analysis.

According to further features of the invention described below, the level of RNA encoded by the gene in blood of the test subject is determined by probing a microarray.

According to further features of the invention described below, the level of RNA encoded by the gene in blood of the test subject and the levels of RNA encoded by the gene in blood of the control subjects are determined via the same method.

In further aspects, there is provided isolated compositions, test systems and primer sets for use in the methods disclosed herein.

In one aspect, there is provided an isolated composition comprising, a blood sample from a test subject and for each gene of a set of one or more genes selected from the genes listed in Table 2, one or more components selected from the group consisting of exogenous RNA encoded by the gene, cDNA complementary to the RNA, an oligonucleotide which specifically hybridizes to the cDNA or the RNA under stringent conditions, a primer set capable of generating an amplification product of the cDNA complementary to RNA, and an amplification product of the cDNA. In one embodiment, the set of genes comprises or consists of an ASGR2 gene and a STAB1 gene.

In another aspect there is provided an isolated composition comprising, for each gene of a set of genes selected from the genes listed in Table 2, one or more components selected from the group consisting of: an exogenous isolated RNA encoded by the gene, cDNA complementary to the RNA, an oligonucleotide which specifically hybridizes to the cDNA or the RNA under stringent conditions, a primer set capable of generating an amplification product of the cDNA complementary to RNA, and an amplification product of the cDNA. In one embodiment, the set of genes comprises or consists of an ASGR2 gene and a STAB1 gene.

In a further aspect, there is provided a primer set comprising a first primer and a second primer, wherein the first primer is one of a set of primers capable of generating an amplification product of cDNA complementary to RNA encoded by a first gene, wherein the second primer is capable of generating an amplification product of cDNA complementary to RNA encoded by a second gene, and wherein the first gene and the second gene are different genes selected from the genes listed in Table 2, or composition thereof. In one embodiment, the set of genes comprises or consists of an ASGR2 gene and a STAB1 gene.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The disclosure will now be described in relation to the drawings in which:

FIG. 1 shows genes differentially regulated in heart failure. A total of 243 unique known genes were identified. The dendrogram was constructed using average linkage as the distance measurement and Pearson correlation as the similarity measurement.

FIG. 2 is a graphical depiction functionally categorizing genes differentially regulated in heart failure.

FIG. 3 shows the pathway of T cell receptor signalling. Heart failure (HF)-regulated genes are marked in grey.

FIG. 4 shows an exemplary computer system.

### DETAILED DESCRIPTION OF THE DISCLOSURE

As will become apparent, preferred features and characteristics of one aspect are applicable to any other aspect. It should be noted that, as used herein, the singular form “a”, “an” and “the” include plural references unless the context clearly dictates otherwise.

The term “encode” as used herein means that a polynucleotide, including a gene, is said to “encode” a RNA and/or polypeptide if, in its native state or when manipulated by methods well known to those skilled in the art, it can be transcribed and/or translated to produce the mRNA for and/or the polypeptide or a fragment thereof. The anti-sense strand is the complement of such a nucleic acid, and the encoding sequence can be deduced there from.

The term “label” as used herein refers to a composition capable of producing a detectable signal indicative of the presence of the target polynucleotide in an assay sample. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

As used herein, a “sample” refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), organs, and also samples of in vitro cell culture constituent.

The term “gene” as used herein is a polynucleotide which may include coding sequences, intervening sequences and regulatory elements controlling transcription and/or translation. Genes of the disclosure include normal alleles of the gene encoding polymorphisms, including silent alleles having no effect on the amino acid sequence of the gene's encoded polypeptide as well as alleles leading to amino acid sequence variants of the encoded polypeptide that do not substantially affect its function. These terms also may optionally include alleles having one or more mutations which affect the function of the encoded polypeptide's function.

The polynucleotide compositions, such as primers, of this disclosure include RNA, cDNA, DNA complementary to target cDNA of this invention or portion thereof, genomic DNA, unspliced RNA, spliced RNA, alternately spliced RNA, synthetic forms, and mixed polymers, both sense and antisense strands, and may be chemically or biochemically modified or may contain non-natural or derivatized nucleotide bases, as will be readily appreciated by those skilled in the art.

Where nucleic acid according to the disclosure includes RNA, reference to the sequence shown should be construed as reference to the RNA equivalent, with U substituted for T.

The term “amount” or “level” of RNA encoded by a gene described herein encompasses the absolute amount of the RNA, the relative amount or concentration of the RNA, as well as any value or parameter which correlates thereto.

The methods of nucleic acid isolation, amplification and analysis are routine for one skilled in the art and examples of protocols can be found, for example, in the Molecular Cloning: A Laboratory Manual (3-Volume Set) Ed. Joseph Sambrook, David W. Russel, and Joe Sambrook, Cold Spring Harbor Laboratory; 3rd edition (Jan. 15, 2001), ISBN: 0879695773. Particularly useful protocol source for methods used in PCR amplification is PCR (Basics: From Background to Bench) by M. J. McPherson, S. G. Moller, R. Beynon, C. Howe, Springer Verlag; 1st edition (Oct. 15, 2000), ISBN: 0387916008.

“Heart failure” as used herein means a condition that impairs the ability of the heart to fill with blood or pump a sufficient amount of blood through the body resulting from a structural or functional cardiac disorder. Heart failure may be interchangeably referred to as congestive heart failure (CHF) or congestive cardiac failure (CCF). Stages of heart failure may be defined using any one of various classification systems known in the art. For example, heart failure may be classified using the New York Heart Association (NYHA) classification system. According to the NYHA classification system, there are 4 main classes of heart failure; NYHA stage I (NYHA I) heart failure, NYHA stage II (NYHA II) heart failure, NYHA stage III (NYHA III) heart failure and NYHA stage IV (NYHA IV) heart failure. These stages classify heart failure according to the following: NYHA I: No symptoms and no limitation in ordinary physical activity; NYHA II: Mild symptoms (mild shortness of breath and/or angina pain) and slight limitation during ordinary activity; NYHA III: Marked limitation in activity due to symptoms, even during less-than-ordinary activity (e.g. walking short distances, about 20 to 100 meters). Comfortable only at rest; NYHA IV: Severe limitations. Symptoms are experienced even while at rest, mostly bedbound patients.

As used herein, “Compensated heart failure” corresponds to NYHA I/NYHA II heart failure.

As used herein, “Decompensated heart failure” corresponds to NYHA III/NYHA IV heart failure.

A “control population” refers to a defined group of individuals or a group of individuals with or without heart failure or with a particular heart failure classification, and may optionally be further identified by, but not limited to geographic, ethnic, race, gender, one or more other conditions or diseases, and/or cultural indices. In most cases a control population may encompass at least 10, 50, 100, 1000, or more individuals.

“Positive control data” encompasses data representing levels of RNA encoded by a target gene of the invention in each of one or more subjects having heart failure or a particular heart failure classification, and encompasses a single data point representing an average level of RNA encoded by a target gene of the invention in a plurality of subjects having heart failure or the particular heart failure classification.

“Negative control data” encompasses data representing levels of RNA encoded by a target gene of the invention in each of one or more subjects not having heart failure, and encompasses a single data point representing an average level of RNA encoded by a target gene of the invention in a plurality of subjects not having heart failure.

The probability that test data “corresponds” to positive control data or negative control data refers to the probability that the test data is more likely to be characteristic of data obtained in subjects having heart failure or the particular heart failure classification than in subjects not having any heart failure or the particular heart failure classification, or is more likely to be characteristic of data obtained in subjects not having any heart failure or the particular heart failure classification than in subjects having heart failure or the particular heart failure classification, respectively.

A gene expression profile for heart failure or a particular heart failure classification found in blood at the RNA level of one or more genes listed in Table 2 or Table 3 can be identified or confirmed using many techniques, including but preferably not limited to PCR methods, as for example discussed further in the working examples herein, Northern analyses and the microarray technique. This gene expression profile can be measured in a bodily sample, such as blood, using microarray technology. In an embodiment of this method, fluorescently labeled cDNA probes may be generated through incorporation of fluorescent nucleotides by reverse transcription of RNA extracted from blood. Labeled cDNA probes applied to the chip hybridize with specificity to each spot of DNA on the array. Quantitation of hybridization of each arrayed element allows for assessment of corresponding mRNA abundance. For example, with dual color fluorescence, separately labeled cDNA probes generated from two sources of RNA are hybridized pair wise to the array. The relative abundance of the transcripts from the two sources corresponding to each specified gene is thus determined simultaneously. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2):106-149 (1996)). Microarray analysis can be performed by commercially available equipment, following manufacturer's protocols, such as by using the Affymetrix GeneChip technology, or Incyte's microarray technology.

### Methods

According to one aspect, there is provided a method of determining a severity of heart failure in a human test subject. The method comprises, for each gene of a set of one or more genes listed in Table 2, a step of providing test data representing a level of RNA encoded by the gene in blood of the test subject and providing positive control data representing levels of RNA encoded by the gene in blood of human control subjects having a categorized severity of heart failure. The method comprises a subsequent step of comparing the level of RNA in blood of the test subject to the levels in blood of control subjects to thereby determine a value indicating whether the test data corresponds to the positive control data, where a correspondence between the test data and the positive control data indicates that the test subject has the categorized severity of heart failure.

In one embodiment, the test data is provided by determining a level of RNA encoded by the gene in blood of the test subject, and/or the positive control data is provided by determining levels of RNA encoded by the gene in blood of human subjects having the categorized severity of heart failure.

In another embodiment, comparing the level of RNA encoded by the gene in blood of the test subject to the levels in blood of control subjects is effected by inputting, to a computer, the test data, where the computer is for comparing data representing a level of RNA encoded by the gene in blood of a human subject to levels of RNA encoded by the gene in subjects having the categorized severity of heart failure, to thereby output a value indicating whether the test data corresponds to the positive control data; and causing the computer to compare the test data to the positive control data, to thereby output the value indicating whether the test data corresponds to the positive control data.

According to another aspect, there is provided a method of determining whether a human test subject has heart failure as opposed to not having heart failure, the method comprising for each gene of a set of one or more of the genes listed in Table 2: (a) determining a level of RNA encoded by the gene in blood of the test subject, thereby generating test data; (b) providing a positive control data representing levels of RNA encoded by the gene in blood of human control subjects having heart failure and a negative control data representing levels of RNA encoded by the gene in blood of human control subject not having heart failure; and (c) comparing the levels of a) and b) to determine whether the test data corresponds to the positive control data or the negative control data; wherein a correspondence between the test data and the positive control data and not the negative control data indicates that the test subject has heart failure.

According to yet another aspect, there is provided a method of determining a severity of heart failure in a human test subject, the method comprising for each gene of a set of one or more of the genes listed in Table 2: (a) determining a level of RNA encoded by the gene in blood of the test subject, thereby generating test data; (b) providing a first positive control data representing levels of RNA encoded by the gene in blood of human control subjects having a first categorized severity of heart failure; and (c) comparing the levels of a) and b) to determine whether the test data corresponds to the positive control data; wherein a correspondence between the test data and the positive control data indicates that the test subject has the first categorized severity of heart failure.

In an embodiment, the set of genes comprises or consists of ASGR2 and STAB1.

In another embodiment, the set of genes comprises or consists of ASGR2, C3AR1 and/or STAB1.

In one embodiment, the first categorized severity is compensated heart failure or decompensated heart failure.

In another embodiment, the method further comprises providing a second positive control data representing levels of RNA encoded by the gene in blood of human control subjects having a second categorized severity of heart failure, wherein correspondence between the test data and the second positive control data indicates that the test subject has the second categorized severity of heart failure. In one embodiment, the first categorized severity is compensated heart failure and the second categorized severity is decompensated heart failure. In such an embodiment, the method allows determination of the likelihood that a particular heart failure patient falls within a compensated heart failure class or a decompensated heart failure class, which is relevant to types of treatment available to the subject.

In an embodiment, the method further comprises determining levels of RNA encoded by the gene in blood of a population of human subjects having the first categorized severity of heart failure, thereby providing the positive control data representing the levels of RNA encoded by the gene in blood of human control subjects having the first categorized severity of heart failure. In yet another embodiment, the method further comprises determining levels of RNA encoded by the gene in blood of a population of human subjects having the second categorized severity of heart failure, thereby providing the positive control data representing the levels of RNA encoded by the gene in blood of human control subjects having the second categorized severity of heart failure.

In a further embodiment, the method further comprises providing a third control data representing levels of RNA encoded by the gene in blood of human control subjects which are healthy, and wherein step c) is effected by comparing the test data to the first or second positive control data and the third control data, wherein correspondence with the first or second positive control data and not the third control data indicates that the test subject has the first or second categorized severity of heart failure.

In a further aspect, there is provided a method of monitoring the progression of heart failure in a human subject, the method comprising for each gene of a set of one or more of ASGR2, C3AR1, STAB1, KRCC1, KYNU, LOH11CR2A, TMEM144, FKBP1B, VCAN, LTA4H, MGST1, or NOTCH2NL (a) determining a level of RNA encoded by the gene in blood of the subject at a first time point; (b) determining a level of RNA encoded by an ASGR2 gene in blood of the subject at a second time point; (c) comparing the levels in a) and b); wherein an increased level at the second time point indicates a progression of heart failure. In an embodiment, the one or more genes comprise or consist of ASGR2, C3AR1 and/or STAB1.

In another aspect, there is provided a method of monitoring the progression of heart failure in a human subject, the method comprising, for each gene of a set of one or more of FAM84B, RBL2, XRCC5, TNRC6C, ZBTB44, HERC1, SNORA72, WIPF1, PPP1R2, C4orf30, KIAA0888, TMEM106B, NR3c2, KLHL24, FLJ31306, MAP2K6, SATB1, WHDC1L1, EDG1, MBIP, RSU1, DYRK2, DOCK9, LOC283666, SLC4A7, ODF2L, PAQR8, or C14orf64 or the genes listed in Table 4 (a) determining a level of RNA encoded by the gene in blood of the subject at a first time point; (b) determining a level of RNA encoded by the gene in blood of the subject at a second time point; (c) comparing the levels in a) and b); wherein an decreased level at the second time point indicates a progression of heart failure.

In a further aspect, there is provided a method of classifying a human test subject as having decompensated heart failure, the method comprising (a) determining a level of RNA encoded by each gene of a set of one or more of ASGR2, C3AR1, STAB1, KRCC1, KYNU, LOH11CR2A, TMEM144, FKBP1B, VCAN, LTA4H, MGST1, or NOTCH2NL in blood of the test subject, thereby generating test data; (b) providing control data representing a level of RNA encoded by the gene in blood of human control subjects not having decompensated heart failure; and (c) comparing the test data to the control data, wherein a determination in step (c) that the level of RNA encoded by the gene in blood of the test subject is higher than the level of RNA encoded by the gene in blood of human control subjects not having decompensated heart failure is used to classify the test subject as having decompensated heart failure.

In yet another aspect, there is provided a method of classifying a human test subject as having decompensated heart failure, the method comprising for each gene of a set of one or more of FAM84B, RBL2, XRCC5, TNRC6C, ZBTB44, HERC1, SNORA72, WIPF1, PPP1R2, C4orf30, KIAA0888, TMEM106B, NR3c2, KLHL24, FLJ31306, MAP2K6, SATB1, WHDC1L1, EDG1, MBIP, RSU1, DYRK2, DOCK9, LOC283666, SLC4A7, ODF2L, PAQR8, or C14orf64 (a) determining a level of RNA encoded by the gene in blood of the test subject, thereby generating test data; (b) providing control data representing a level of RNA encoded by the gene in blood of human control subjects not having decompensated heart failure; and (c) comparing the test data to the control data, wherein a determination in step (c) that the level of RNA encoded by the gene in blood of the test subject is lower than the level of RNA encoded by the gene in blood of human control subjects not having decompensated heart failure is used to classify the test subject as having decompensated heart failure.

Determining whether the level of RNA of a gene in blood of the test subject is higher than the level of RNA encoded by the gene in blood of control subjects not having heart failure or in the same subject at a different time point may be effected by determining whether there is a fold-change in the level between the test subject and the control subject or different time point which is higher than a minimum fold-change and/or which is within a range of fold-changes.

Determining whether the level of RNA of a gene in blood of the test subject is lower than the level of RNA encoded by the gene in blood of control subjects not having heart failure or in the same subject at a different time point may be effected by determining whether there is a fold-change in the level between the test subject and the control subject or different time point which is lower than a maximum fold-change and/or which is within a range of fold-changes.

For levels of RNA encoded by a given gene, to classify a test subject as NYHA I-II, a suitable minimum fold-change is the fold-change value corresponding to NYHA I-II/control set forth in Table 2, and a suitable range of fold-changes is the fold-change value corresponding to NYHA I-II/control set forth in Table 2 to the fold-change value corresponding to NYHA III-IV/control set forth in Table 2, where control corresponds to an average level of RNA encoded by the gene in blood of healthy subjects. To classify a test subject as NYHA III-IV, a suitable minimum fold-change value is the fold-change value greater than or equal to the NYHA-III-IV/control set forth in Table 2.

Examples of suitable fold-changes and ranges of fold-changes for classifying a test subject are provided in Table 2, and include the following ones. The methods recited in the above and below paragraphs can be done with “about” the cited amounts.

For levels of RNA encoded by ASGR2, to classify a test subject as NYHA-I-II, a suitable minimum fold-change is 1.5 fold, and a suitable range of fold-changes is 1.59 to 2.45 fold, relative to an average level of RNA encoded by the gene in blood of healthy subjects. To classify a test subject as NYHA-III-IV, a suitable minimum fold-change is greater than or equal to 2.45, relative to an average level of RNA encoded by the gene in blood of healthy subjects.

For levels of RNA encoded by C3AR1, to classify a test subject as NYHA-I-II, a suitable minimum fold-change is 1.05 fold, and a suitable range of fold-changes is 1.05 to 1.95 fold, relative to an average level of RNA encoded by the gene in blood of healthy subjects. To classify a test subject as NYHA-III-IV, a suitable minimum fold-change is greater than or equal to 1.95, relative to an average level of RNA encoded by the gene in blood of healthy subjects.

For levels of RNA encoded by STAB1, to classify a test subject as NYHA-I-II, a suitable minimum fold-change is 1.33 fold, and a suitable range of fold-changes is 1.33 to 1.92 fold, relative to an average level of RNA encoded by the gene in blood of healthy subjects. To classify a test subject as NYHA-III-IV, a suitable minimum fold-change is greater than or equal to 1.92, relative to an average level of RNA encoded by the gene in blood of healthy subjects.

As used herein, the term “about” refers to a variability of plus or minus 10 percent.

Thus, a test subject is classified or determined as having or being more likely to have heart failure or a particular heart failure classification than to not have it if, for each marker gene of the particular set of marker genes used to practice the method of classifying or determining, the fold-change in level of RNA encoded by that gene in blood of the test subject relative to blood of the control subjects not having heart failure or the particular heart failure classification, classifies or determines that the test subject has or is more likely to have heart failure or the particular heart failure classification than to not have it.

Conversely, a test subject of the invention is classified or determined as having or being more likely to not have heart failure or the particular heart failure classification if, for each marker gene of the particular set of marker genes used to practice the method of classifying or determining, the fold-change in level of RNA encoded by that gene in blood of the test subject relative to blood of the control subjects does not classify or determine the test subject as having or being more likely to have heart failure or the particular heart failure classification than to not have it.

In one aspect, the set of one or more heart failure marker genes may consist of any one of the possible combinations of one or more of the genes set out in Table 2. In an embodiment, the one or more heart failure marker genes comprise or consist of ASGR2, C3AR1 and/or STAB1.

In a further aspect, there is provided a method of determining whether a human subject with heart failure has a prognosis of mortality, the method comprising for each gene of a set of one or more of the genes set forth in Table 3 (a) determining a level of RNA encoded by the gene in blood of the test subject, thereby generating test data; (b) providing positive control data representing levels of RNA encoded by the gene in blood of human control subjects having a prognosis of mortality; and (c) comparing the levels of a) to b), wherein a correspondence between the test data and the positive control data indicates that the test subject has a prognosis of mortality. In one embodiment, the determining whether the test data corresponds to the positive control data is effected by applying to the test data a mathematical model derived from the positive control data, and wherein the mathematical model is for determining the whether a level of RNA encoded by the gene corresponds to the positive control data. In an embodiment, the one or more heart failure marker genes comprise or consist of FAM134B, MGAT4A, ZCCHC14 or CD28

In yet a further aspect, there is provided a method of ranking two or more test subjects having heart failure according to risk of death, the method comprising for each gene of a set of one or more of the genes set forth in Table 3: (a) determining a level of RNA encoded by the gene in blood of each test subject, thereby generating test data; (b) calculating the risk score for each test subject based on the level of expression in (a); (c) ranking the risk scores of the test subjects, wherein the test subjects are ranked according to risk of death.

In one embodiment, the gene is FAM134B and the equation for calculating the relative risk for this gene is 0.192̂Expression. In another embodiment, the gene is MGAT4A and the equation for calculating the relative risk for this gene is 0.206̂Expression. In yet another embodiment, the gene is ZCCHC14 and the relative risk for this gene is 0.440̂Expression. In a further embodiment, the gene is CD28 and the equation for calculating the relative risk for this gene is 0.451̂Expression. “Expression” in the relative risk equations refers to blood RNA levels in log scale for the gene in a test subject, determined, e.g. as described in the Materials and Methods. These equations were derived using the Cox method described herein. The symbol “̂” indicates, according to convention, that the indicated gene-specific numerical coefficient is raised to an exponent corresponding to the value of the RNA level.

In an aspect of the invention, the level of RNA encoded by the gene in blood of the test subject and/or the levels in blood of positive control subjects are relative to a level of RNA encoded by the gene in blood of healthy test subjects. Thus, in one embodiment, the level of RNA encoded by the gene in blood of the test subject is determined as a ratio to a level of RNA encoded by the gene in blood of a healthy test subject. Thus, in another embodiment, the levels of RNA encoded by the gene in blood of control subjects are determined as a ratio to a level of RNA encoded by the gene in blood of a healthy test subject.

It will be appreciated that data representing levels of RNA encoded by a set of genes of the disclosure may be combined with data representing levels of gene products of other genes which are differently expressed in blood in subjects having heart failure relative to subjects not having any heart failure so as to determine a probability that a test subject has heart failure versus not having heart failure, or for the purposes of classifying the stage of heart failure.

In another aspect, the method further comprises determining levels of RNA encoded by the gene in blood of a population of control human subjects having heart failure, and/or in blood of a population of human control subjects not having heart failure, to thereby provide the positive control data and/or the negative control data, respectively. Alternately, it is envisaged that the level of RNA encoded by a gene of the invention in control subjects of the invention could be provided by prior art data corresponding to control data. In one embodiment, there is provided a first positive control data derived from subjects having a first categorized severity of heart disease, optionally, compensated or decompensated heart failure. In another embodiment, there is a first and second positive control data and the first positive control data is derived from subjects having compensated heart failure and the second positive control data is derived from subjects having decompensated heart failure.

The method may be practiced using any one of various types of control subjects.

In an aspect, the control subjects not having heart failure are subjects having been diagnosed as not having any heart failure as a result of routine examination. As is described in the Examples section which follows, the method of the invention may be practiced using subjects not having heart failure as the control subjects not having heart failure.

The methods described herein may furthermore be practiced using any one of various numbers of control subjects. One of ordinary skill in the art will possess the necessary expertise to select a sufficient number of control subjects so as to obtain control data having a desired statistical significance for practicing the method of the invention with a desired level of reliability.

For example, the method can be practiced using 10 or more, 20 or more, 30 or more, 40 or more, 50 or more, 60 or more, 70 or more, 80 or more, 90 or more, 100 or more, 10 or more, 20 or more, 30 or more, 40 or more, 50 or more, 60 or more, 70 or more, 80 or more, 90 or more, 100 or more, 110 or more, 120 or more, 130 or more, 140 or more, 150 or more, 160 or more, 170 or more, 180 or more, 190 or more, or 200 or more of control subjects having heart failure and/or a particular classification of heart failure and/or of control subjects not having heart failure.

In one aspect of the invention, the level of RNA encoded by a gene in blood of the test subject and the levels of RNA encoded by the gene in blood of the control subjects are determined via the same method. As is described in the Examples section, below, the method can be practiced where the level of RNA encoded by a gene in blood of the test subject and the levels of RNA encoded by the gene in blood of the control subjects are determined via the same method. Alternately, it is envisaged that the level of a gene in blood of a test subject of the invention and in blood of control subjects of the invention could be determined using different methods. It will be appreciated that use of the same method to determine the levels of RNA encoded by a gene of the invention in a test subject and in control subjects can be used to avoid method-to-method calibration to minimize any variability which might arise from use of different methods.

In one aspect, determining of the level of RNA encoded by a gene of the invention in blood of a subject of the invention is effected by determining the level of RNA encoded by the gene in a blood sample isolated from the subject. Alternately, it is envisaged that determination of the level of RNA encoded by the gene in blood of a subject of the invention could be effected by determining the level of RNA encoded by the gene in an in-vivo sample using a suitable method for such a purpose.

In one aspect, the level of RNA encoded by a gene in blood of a subject is determined in a sample of RNA isolated from blood of the subject.

Alternately, it is envisaged that the level of RNA of a gene in blood of a subject could be determined in a sample which includes RNA of blood of the subject but from which RNA has not been isolated therefrom, using a suitable method for such a purpose.

Any one of various methods routinely employed in the art for isolating RNA from blood may be used to isolate RNA from blood of a subject, so as to enable practicing of the methods described herein.

In one aspect, the level of RNA encoded by a gene in blood of a subject is determined in RNA of a sample of whole blood. Any one of various methods routinely employed in the art for isolating RNA from whole blood may be employed for practicing the method.

Alternately, it is envisaged that the level of RNA encoded by a gene in blood of a subject could be determined in RNA of a sample of fraction of blood which expresses the gene sufficiently specifically so as to enable the method. Examples of such blood fractions include preparations of isolated types of leukocytes, preparations of isolated peripheral blood mononuclear cells, preparations of isolated granulocytes, preparations of isolated whole leukocytes, preparations of isolated specific types of leukocytes, plasma-depleted blood, preparations of isolated lymphocytes, and the plasma fraction of blood.

In one aspect of the method, isolation of RNA from whole blood of a subject of the invention is effected using EDTA tubes, as described in the Examples section.

In another aspect of the method, isolation of RNA from whole blood of a subject of the invention may be effected by using a PAXgene Blood RNA Tube (obtainable from PreAnalytiX) in accordance with the instructions of the PAXgene Blood RNA Kit protocol.

Determination of a level of RNA encoded by a gene in a sample of the invention may be effected in any one of various ways routinely practiced in the art.

For example, the level of RNA encoded by a gene in a sample may be determined via any one of various methods based on quantitative polynucleotide amplification which are routinely employed in the art for determining a level of RNA encoded by a gene in a sample.

Alternatively, the level of RNA encoded by a gene may be determined via any one of various methods based on quantitative polynucleotide hybridization to an immobilized probe which are routinely employed in the art for determining a level of RNA encoded by a gene in a sample.

In one aspect of the methods described herein, quantitative polynucleotide amplification used to determine the level of RNA encoded by a gene is quantitative reverse transcriptase-polymerase chain reaction (PCR) analysis. Any one of various types of quantitative reverse transcriptase-PCR analyses routinely employed in the art to determine the level of RNA encoded by a gene in a sample may be used to practice the methods. For example, any one of various sets of primers may be used to perform quantitative reverse transcriptase-PCR analysis so as to practice the methods.

In one aspect, the quantitative reverse transcriptase-PCR analysis used to determine the level of RNA encoded by a gene is quantitative real-time PCR analysis of DNA complementary to RNA encoded by the gene using a labeled probe capable of specifically binding amplification product of DNA complementary to RNA encoded by the gene. For example, quantitative real-time PCR analysis may be performed using a labeled probe which comprises a polynucleotide capable of selectively hybridizing with a sense or antisense strand of amplification product of DNA complementary to RNA encoded by the gene. Labeled probes comprising a polynucleotide having any one of various nucleic acid sequences capable of specifically hybridizing with amplification product of DNA complementary to RNA encoded by the gene may be used to practice the methods described herein.

Quantitative real-time PCR analysis of a level of RNA encoded by a gene may be performed in any one of various ways routinely employed in the art.

In one aspect, quantitative real-time PCR analysis is performed by analyzing complementary DNA prepared from RNA of blood a subject of the invention, using the QuantiTect™ Probe RT-PCR system (Qiagen, Valencia, Calif.; Product Number 204345), a TaqMan dual labelled probe, and a Real-Time PCR System 7500 instrument (Applied Biosystems).

As specified above, the level of RNA encoded by a gene may be determined via a method based on quantitative polynucleotide hybridization to an immobilized probe.

In one aspect, determination of the level of RNA encoded by a gene via a method based on quantitative polynucleotide hybridization is effected using a microarray, such as an Affymetrix U133Plus 2.0 GeneChip oligonucleotide array (Affymetrix; Santa Clara, Calif.).

As specified above, the level of RNA encoded by a gene in a sample of the invention may be determined via quantitative reverse transcriptase-PCR analysis using any one of various sets of primers and labeled probes to amplify and quantitate DNA complementary to RNA encoded by a marker gene produced during such analysis. Examples of suitable primers for use in quantitative reverse transcriptase-PCR analysis of the level of RNA encoded by a target gene are within the knowledge of a person skilled in the art.

In one aspect, the primers may be selected so as to include a primer having a nucleotide sequence which is complementary to a region of a target cDNA template, where the region spans a splice junction joining a pair of exons. It will be appreciated that such a primer can be used to facilitate amplification of DNA complementary to messenger RNA, i.e. mature spliced RNA.

It will be appreciated that the probability that the test subject does not have any heart failure as opposed to having heart failure can be readily determined from the probability that the test subject has heart failure as opposed to not having heart failure. For example, when expressing the probability that the test subject has heart failure as a percentage probability, the probability that the test subject does not have any heart failure as opposed to having heart failure corresponds to 100 percent minus the probability that the test subject does not have any heart failure as opposed to having heart failure.

Determining the probability that the test data corresponds to positive control data and not to the negative control data may be effected in any one of various ways known to the ordinarily skilled artisan for determining the probability that a gene expression profile of a test subject corresponds to a gene expression profile of subjects having a pathology and not to a gene expression profile of subjects not having the pathology, where the gene expression profiles of the subjects having the pathology and the subjects not having the pathology are significantly different.

In one aspect of the method, determining the probability that the test data corresponds to the positive control data and not to the negative control data is effected by applying to the test data a mathematical model derived from the positive control data and from the negative control data.

In another aspect, determining whether the test data corresponds to positive control data may be effected in any one of various ways known to the ordinarily skilled artisan for determining whether a gene expression profile of a test subject corresponds to a gene expression profile of subjects having a pathology, where the gene expression profiles of the subjects having the pathology and the subjects not having the pathology are significantly different.

In one aspect, determining whether the test data corresponds to the positive control data is effected by applying to the test data a mathematical model derived from the positive control data.

Various suitable mathematical models which are well known in the art of medical diagnosis using disease markers may be employed to compare test data to control data so as to classify, according to the present teachings, a test subject as more likely to have or having heart failure or a particular heart failure classification than to not have heart failure or the particular classification, to determine a probability that a test subject is likely to have heart failure or a particular heart failure classification as opposed to not having heart failure or the particular classification, or to diagnose a test subject as having colorectal cancer according to the teachings described herein. Generally these mathematical models can be unsupervised methods performing a clustering whilst supervised methods are more suited to classification of datasets. (refer, for example, to: Dreiseitl S, Ohno-Machado L. Logistic regression and artificial neural network classification models: a methodology review. J Biomed Inform. 2002 October-December; 35(5-6):352-9; Pepe M S. The Statistical Evaluation of Medical Tests for Classification and Prediction. Oxford, England: Oxford University Press; 2003; Dupont WD. Statistical Modeling for Biomedical Researchers. Cambridge, England: Cambridge University Press; 2002; Pampel FC. Logistic regression: A Primer. Publication # 07-132, Sage Publications: Thousand Oaks, Calif. 2000; King E N, Ryan T P. A preliminary investigation of maximum likelihood logistic regression versus exact logistic regression. Am Statistician 2002; 56:163-170; Metz C E. Basic principles of ROC analysis. Semin Nucl Med 1978; 8:283-98; Swets J A. Measuring the accuracy of diagnostic systems. Science 1988; 240:1285-93; Zweig M H, Campbell G. Receiver-operating characteristic (ROC) plots: a fundamental evaluation tool in clinical medicine. Clin Chem 1993; 39:561-77; Witten I H, Frank Eibe. Data Mining: Practical Machine Learning Tools and Techniques (second edition). Morgan Kaufman 2005; Deutsch J M. Evolutionary algorithms for finding optimal gene sets in microarray prediction. Bioinformatics 2003; 19:45-52; Niels Landwehr, Mark Hall and Eibe Frank (2003) Logistic Model Trees. pp 241-252 in Machine Learning: ECML 2003: 14th European Conference on Machine Learning, Cavtat-Dubrovnik, Croatia, Sep. 22-26, 2003, Proceedings Publisher: Springer-Verlag GmbH, ISSN: 0302-9743). Examples of such mathematical models, related to learning machine, include: Random Forests methods, logistic regression methods, neural network methods, k-means methods, principal component analysis methods, nearest neighbour classifier analysis methods, linear discriminant analysis, methods, quadratic discriminant analysis methods, support vector machine methods, decision tree methods, genetic algorithm methods, classifier optimization using bagging methods, classifier optimization using boosting methods, classifier optimization using the Random Subspace methods, projection pursuit methods, genetic programming and weighted voting methods.

### Computer-Based Methods

It will be appreciated that a computer may be used for determining the probability that the test subject has heart failure or a particular classification using a mathematical model, according to the methods described herein.

Thus, according to another aspect of the invention there is provided a computer-based method of determining a severity of heart failure in a human test subject. Accordingly, there is provided a computer-based method of determining a severity of heart failure in a human test subject, the method comprising inputting, to a computer, test data representing a level of RNA encoded by one or more of the genes listed in Table 2 in blood of the test subject; and causing the computer to compare the test data to a first positive control data representing levels of RNA encoded by the gene in blood of human control subjects having a first categorized severity of heart failure, wherein correspondence between the test data and the first positive control data indicates that the test subject has the first categorized severity of heart failure. In one embodiment, the one or more genes is ASGR2, C3AR1 and/or STAB1.

In another aspect, there is provided computer-based method of monitoring the progression of heart failure in a human subject. Accordingly, there is provided a computer-based method of monitoring the progression of heart failure in a human subject, the method comprising inputting, to a computer, test data representing a level of RNA encoded by one or more of ASGR2, C3AR1, STAB1, KRCC1, KYNU, LOH11CR2A, TMEM144, FKBP1B, VCAN, LTA4H, MGST1, or NOTCH2NL gene in blood of the subject at a first and second time point; and causing the computer to compare the data of the first time point to the data of the second time point, and to determine whether the level of RNA encoded by the gene in blood of the subject is increased at the second time point compared to the level of RNA encoded by the gene in blood of the subject at the first time point, wherein a determination that the level of RNA encoded by the gene in blood of the test subject is increased at the second time point indicates the progression of heart failure. In an additional aspect, there is provided a computer-based method of monitoring the progression of heart failure in a human subject, the method comprising inputting, to a computer, test data representing a level of RNA encoded by one or more of FAM84B, RBL2, XRCC5, TNRC6C, ZBTB44, HERC1, SNORA72, WIPF1, PPP1R2, C4orf30, KIAA0888, TMEM106B, NR3c2, KLHL24, FLJ31306, MAP2K6, SATB1, WHDC1L1, EDG1, MBIP, RSU1, DYRK2, DOCK9, LOC283666, SLC4A7, ODF2L, PAQR8, or C14orf64 gene or the genes listed in Table 4 in blood of the subject at a first and second time point; and causing the computer to compare the data of the first time point to the data of the second time point, and to determine whether the level of RNA encoded by the gene in blood of the subject is decreased at the second time point compared to the level of RNA encoded by the gene in blood of the subject at the first time point, wherein a determination that the level of RNA encoded by the gene in blood of the test subject is decreased at the second time point indicates the progression of heart failure.

According to another aspect of the invention there is provided a computer-based method of classifying a human test subject as having decompensated heart failure. Accordingly, there is provided a computer-based method of classifying a human test subject as having decompensated heart failure, the method comprising for each gene of a set of one or more of ASGR2, C3AR1, STAB1, KRCC1, KYNU, LOH11CR2A, TMEM144, FKBP1B, VCAN, LTA4H, MGST1, or NOTCH2NL inputting, to a computer, test data representing a level of RNA encoded by a STAB1 gene in blood of the test subject; and causing the computer to compare the test data to control data representing a level of RNA encoded by the gene in blood of human control subjects not having decompensated heart failure, and to determine whether the level of RNA encoded by the gene in blood of the test subject is higher than the level of RNA encoded by the gene in blood of human control subjects not having decompensated heart failure, wherein a determination that the level of RNA encoded by the gene in blood of the test subject is higher than the level of RNA encoded by the gene in blood of human control subjects not having decompensated heart failure is used to classify the test subject as having decompensated heart failure. In yet a further aspect, there is provided a computer-based method of classifying a human test subject as having decompensated heart failure, the method comprising for each gene of a set of one or more of FAM84B, RBL2, XRCC5, TNRC6C, ZBTB44, HERC1, SNORA72, WIPF1, PPP1R2, C4orf30, KIAA0888, TMEM106B, NR3c2, KLHL24, FLJ31306, MAP2K6, SATB1, WHDC1L1, EDG1, MBIP, RSU1, DYRK2, DOCK9, LOC283666, SLC4A7, ODF2L, PAQR8, or C14orf64 inputting, to a computer, test data representing a level of RNA encoded by the gene in blood of the test subject; and causing the computer to compare the test data to control data representing a level of RNA encoded by the gene in blood of human control subjects not having decompensated heart failure, and to determine whether the level of RNA encoded by the gene in blood of the test subject is lower than the level of RNA encoded by the gene in blood of human control subjects not having decompensated heart failure, wherein a determination that the level of RNA encoded by the gene in blood of the test subject is lower than the level of RNA encoded by the gene in blood of human control subjects not having decompensated heart failure is used to classify the test subject as having decompensated heart failure.

In yet another aspect, there is provided a computer-based method of determining whether a human test subject with heart failure has a prognosis of mortality. Accordingly, there is provided a computer-based method for determining whether a human subject with heart failure has a prognosis of mortality, the method comprising inputting, to a computer, test data representing a level of RNA encoded by one or more of the genes set forth in Table 3 in blood of the test subject; and causing the computer to compare the test data to a positive control data representing levels of RNA encoded by the gene in blood of human control subjects having a prognosis of mortality, wherein correspondence between the test data and the positive control data indicates that the test subject has the prognosis of mortality. In one embodiment, the one or more genes is FAM134B, MGAT4A, ZCCHC14 or CD28.

In yet a further aspect, there is provided a computer-based method of ranking two or more test subjects having heart failure according to risk of death, the method comprising for each gene of a set of one or more of the genes set forth in Table 3: inputting, to a computer, test data representing a level of RNA encoded by the gene in blood of each test subject; causing the computer to apply the test data to a relative risk equation for assigning a risk score to a test subject based on the level of RNA; and causing the computer to rank the risk score of the test subjects, thereby ranking the test subjects according to risk of death.

In one embodiment, the gene is FAM134B and the equation for calculating the relative risk for this gene is 0.192̂Expression. In another embodiment, the gene is MGAT4A and the equation for calculating the relative risk for this gene is 0.206̂Expression. In yet another embodiment, the gene is ZCCHC14 and the relative risk for this gene is 0.440̂Expression. In a further embodiment, the gene is CD28 and the equation for calculating the relative risk for this gene is 0.451̂Expression. “Expression” in the relative risk equations refers to the log of blood RNA levels for the gene in a test subject, determined, e.g. as described in the Materials and Methods. These equations were derived using the Cox method described herein. The symbol “̂” indicates, according to convention, that the indicated gene-specific numerical coefficient is raised to an exponent corresponding to the value of the RNA level.

Application of computers for determining a probability or whether a test subject has a disease as opposed to not having the disease, so as to enable the method, is routinely practiced in the art using computer systems, and optionally computer-readable media, routinely used in the art.

Thus, according to a further aspect of the invention there is provided a computer system for providing the probability or determining that the test subject has heart failure or a particular classification as opposed to not having heart failure or the particular classification. The computer system comprises a processor; and a memory configured with instructions that cause the processor to provide a user with the probability or answer, where the instructions comprise applying a mathematical model to test data, to thereby determine the probability or whether the test subject has heart failure or the particular classification as opposed to not having heart failure or the particular classification.

The instructions may be provided to the computer in any one of various ways routinely employed in the art. In one aspect, the instructions are provided to the computer using a computer-readable medium.

Thus, according to yet another aspect of the invention there is provided a computer-readable medium having instructions stored thereon that are operable when executed by a computer for applying a mathematical model to test data, thereby determine the probability or whether a test subject has heart failure or the particular classification as opposed to not having heart failure or the particular classification.

As described above, following the step of obtaining the test data, the method of classifying of the invention comprises the step of comparing test data representing a level of RNA encoded by a marker gene to positive control data and/or negative control data, and determining the fold-change between the levels.

It will be appreciated that a computer may be used for comparing test data representing a level of RNA encoded by a marker gene to positive control data and/or negative control data, and determining the fold-change between the levels, according to methods of the invention.

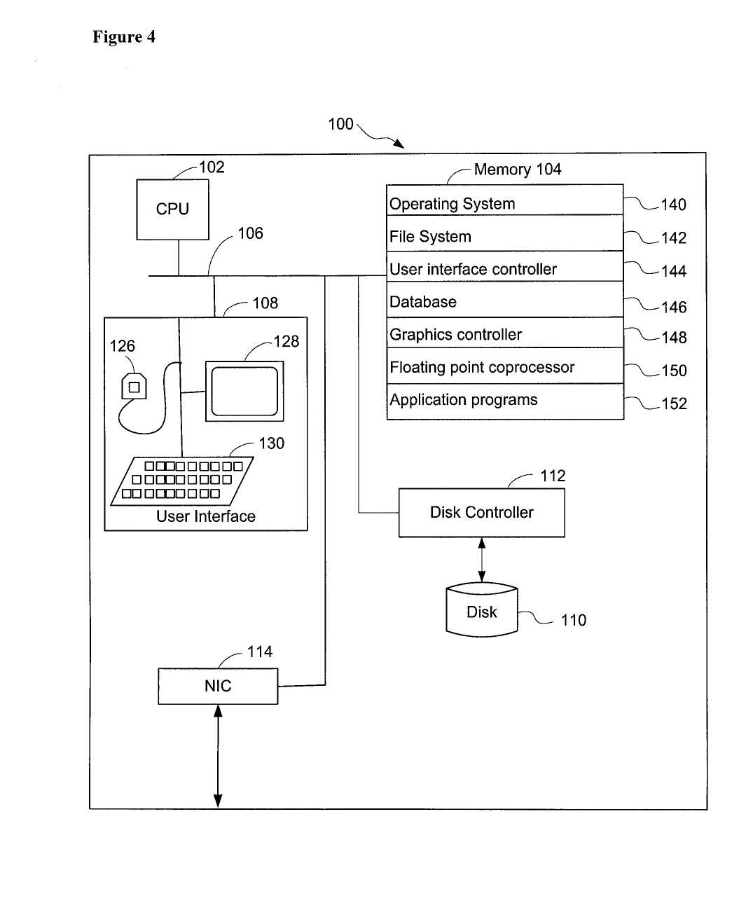

An exemplary computer system for practicing certain of the methods described herein is described in FIG. 4.

FIG. 4 shows a schematic of a general-purpose computer system 100 suitable for practicing the methods described herein. The computer system 100, shown as a self-contained unit but not necessarily so limited, comprises at least one data processing unit (CPU) 102, a memory 104, which will typically include both high speed random access memory as well as non-volatile memory (such as one or more magnetic disk drives) but may be simply flash memory, a user interface 108, optionally a disk 110 controlled by a disk controller 112, and at least one optional network or other communication interface card 114 for communicating with other computers as well as other devices. At least the CPU 102, memory 104, user interface 108, disk controller where present, and network interface card, communicate with one another via at least one communication bus 106.

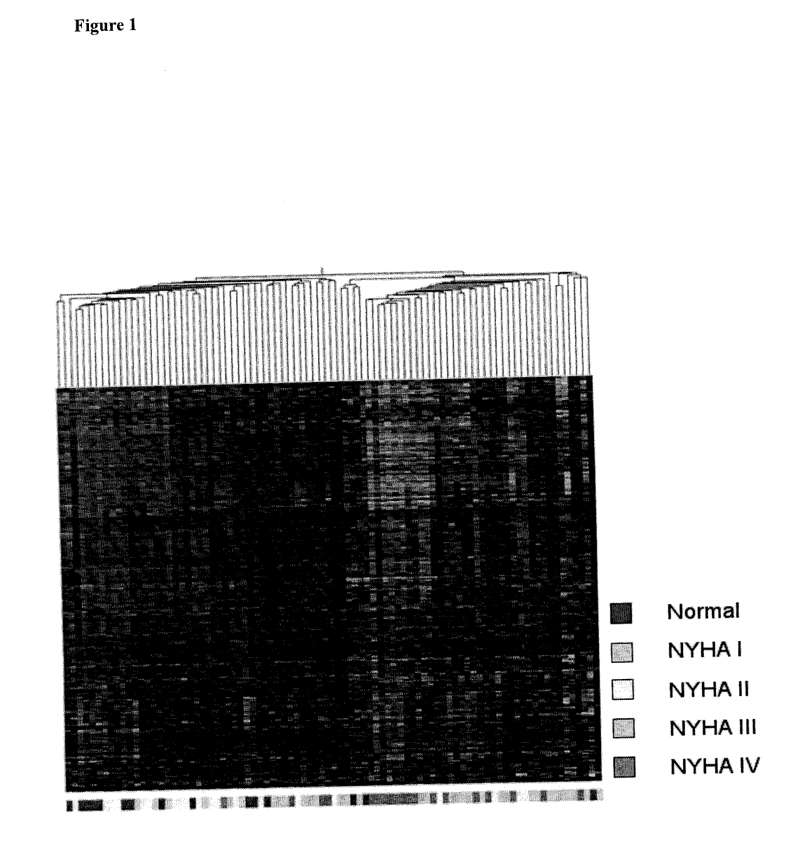

Memory 104 stores procedures and data, typically including: an operating system 140 for providing basic system services; application programs 152 such as user level programs for viewing and manipulating data, evaluating formulae for the purpose of diagnosing a test subject; authoring tools for assisting with the writing of computer programs; a file system 142, a user interface controller 144 for handling communications with a user via user interface 108, and optionally one or more databases 146 for storing data of the invention and other information, optionally a graphics controller 148 for controlling display of data, and optionally a floating point coprocessor 150 dedicated to carrying out mathematical operations. The methods of the invention may also draw upon functions contained in one or more dynamically linked libraries, not shown in FIG. 1, but stored either in Memory 104, or on disk 110, or accessible via network interface connection 114.

User interface 108 may comprise a display 128, a mouse 126, and a keyboard 130. Although shown as separate components in FIG. 1, one or more of these user interface components can be integrated with one another in embodiments such as handheld computers. Display 128 may be a cathode ray tube (CRT), or flat-screen display such as an LCD based on active matrix or TFT embodiments, or may be an electroluminescent display, based on light emitting organic molecules such as conjugated small molecules or polymers. Other embodiments of a user interface not shown in FIG. 1 include, e.g., several buttons on a keypad, a card-reader, a touch-screen with or without a dedicated touching device, a trackpad, a trackball, or a microphone used in conjunction with voice-recognition software, or any combination thereof, or a security-device such as a fingerprint sensor or a retinal scanner that prohibits an unauthorized user from accessing data and programs stored in system 100.

System 100 may also be connected to an output device such as a printer (not shown), either directly through a dedicated printer cable connected to a serial or USB port, or wirelessly, or via a network connection.

The database 146 may instead, optionally, be stored on disk 110 in circumstances where the amount of data in the database is too great to be efficiently stored in memory 104. The database may also instead, or in part, be stored on one or more remote computers that communicate with computer system 100 through network interface connection 114.

The network interface 134 may be a connection to the internet or to a local area network via a cable and modem, or ethernet, firewire, or USB connectivity, or a digital subscriber line. Preferably the computer network connection is wireless, e.g., utilizing CDMA, GSM, or GPRS, or bluetooth, or standards such as 802.11a, 802.11b, or 802.11g.

It would be understood that various embodiments and configurations and distributions of the components of system 10 across different devices and locations are consistent with practice of the methods described herein. For example, a user may use a handheld embodiment that accepts data from a test subject, and transmits that data across a network connection to another device or location wherein the data is analyzed according to a formulae described herein. A result of such an analysis can be stored at the other location and/or additionally transmitted back to the handheld embodiment. In such a configuration, the act of accepting data from a test subject can include the act of a user inputting the information. The network connection can include a web-based interface to a remote site at, for example, a healthcare provider. Alternatively, system 10 can be a device such as a handheld device that accepts data from the test subject, analyzes the data, such as by inputting the data into a formula as further described herein, and generating a result that is displayed to the user. The result can then be, optionally, transmitted back to a remote location via a network interface such as a wireless interface. System 100 may further be configured to permit a user to transmit by e-mail results of an analysis directly to some other party, such as a healthcare provider, or a diagnostic facility, or a patient.

### Kits and Compositions

It will be appreciated that components for practicing the methods described herein may be assembled in a kit.

“Kit” refers to a combination of physical elements, e.g., probes, including without limitation specific primers, labeled nucleic acid probes, antibodies, protein-capture agent(s), reagent(s), instruction sheet(s) and other elements useful to practice the invention, in particular to identify the levels of particular RNA molecules in a sample. These physical elements can be arranged in any way suitable for carrying out the invention. For example, probes and/or primers can be provided in one or more containers or in an array or microarray device.

In the context of this disclosure, the term “probe” refers to a molecule which can detectably distinguish between target molecules differing in structure, such as allelic variants. Detection can be accomplished in a variety of different ways but preferably is based on detection of specific binding. Examples of such specific binding include antibody binding and nucleic acid probe hybridization.

The present disclosure encompasses the use of diagnostic kits based on a variety of methodologies, e.g., PCR, reverse transcriptase-PCR, quantitative PCR, microarray, chip, mass-spectroscopy, which are capable of detecting RNA levels in a sample. There is also provided an article of manufacturing comprising packaging material and an analytical agent contained within the packaging material, wherein the analytical agent can be used for determining and/or comparing the levels of RNA encoded by one or more target genes of the disclosure, and wherein the packaging material comprises a label or package insert which indicates that the analytical agent can be used to identify levels of RNA that correspond to a probability that a test subject has heart failure, or to the severity of heart failure or to survival outcome, for example, a probability that the test subject has heart failure as opposed to not having heart failure.

Therefore, there is provided kits comprising degenerate primers to amplify polymorphic alleles or variants of target genes of the invention, and instructions comprising an amplification protocol and analysis of the results.

The kit may alternatively also comprise buffers, enzymes, and containers for performing the amplification and analysis of the amplification products. The kit may also be a component of a screening or prognostic kit comprising other tools such as DNA microarrays. The kit may also provides one or more control templates, such as nucleic acids isolated from sample of patients without heart failure or a categorized severity thereof, and/or nucleic acids isolated from samples of patients with heart failure or a categorized severity thereof.

The kit may also include instructions for use of the kit to amplify specific targets on a solid support. Where the kit contains a prepared solid support having a set of primers already fixed on the solid support, e.g. for amplifying a particular set of target polynucleotides, the kit also includes reagents necessary for conducting a PCR on a solid support, for example using an in situ-type or solid phase type PCR procedure where the support is capable of PCR amplification using an in situ-type PCR machine. The PCR reagents, included in the kit, include the usual PCR buffers, a thermostable polymerase (e.g. Taq DNA polymerase), nucleotides (e.g. dNTPs), and other components and labeling molecules (e.g. for direct or indirect labeling). The kits can be assembled to support practice of the PCR amplification method using immobilized primers alone or, alternatively, together with solution phase primers.

In one embodiment, the kit provides one or more primer pairs, each pair capable of amplifying RNA encoded by a target gene of the invention, thereby providing a kit for analysis of RNA expression of several different target genes of the invention in a biological sample in one reaction or several parallel reactions. Primers in the kits may be labeled, for example fluorescently labeled, to facilitate detection of the amplification products and consequent analysis of the RNA levels.

Examples of amplification techniques include strand displacement amplification, as disclosed in U.S. Pat. No. 5,744,311; transcription-free isothermal amplification, as disclosed in U.S. Pat. No. 6,033,881; repair chain reaction amplification, as disclosed in WO 90/01069; ligase chain reaction amplification, as disclosed in European Patent Appl. 320 308; gap filling ligase chain reaction amplification, as disclosed in U.S. Pat. No. 5,427,930; and RNA transcription-free amplification, as disclosed in U.S. Pat. No. 6,025,134.

In one embodiment, levels of RNA encoded by more than one target gene can be determined in one analysis. A combination kit may therefore include primers capable of amplifying cDNA derived from RNA encoded by different target genes. The primers may be differentially labeled, for example using different fluorescent labels, so as to differentiate between RNA from different target genes.

Multiplex, such as duplex, real-time RT-PCR enables simultaneous quantification of 2 targets in the same reaction, which saves time, reduces costs, and conserves samples. These advantages of multiplex, real-time RT-PCR make the technique well-suited for high-throughput gene expression analysis. Multiplex qPCR assay in a real-time format facilitates quantitative measurements and minimizes the risk of false-negative results. It is essential that multiplex PCR is optimized so that amplicons of all samples are compared in sub-plateau phase of PCR. Yun, Z., I. Lewensohn-Fuchs, P. Ljungman, L. Ringholm, J. Jonsson, and J. Albert. 2003. A real-time TaqMan PCR for routine quantitation of cytomegalovirus DNA in crude leukocyte lysates from stem cell transplant patients. J. Virol. Methods 110:73-79. Yun, Z., I. Lewensohn-Fuchs, P. Ljungman, and A. Vahlne. 2000. Real-time monitoring of cytomegalovirus infections after stem cell transplantation using the TaqMan polymerase chain reaction assays. Transplantation 69:1733-1736. [PubMed]. Simultaneous quantification of up to 2, 3, 4, 5, 6, 7, and 8 or more targets may be useful.

Accordingly, there is provided a kit comprising packaging and containing, for each gene of a set of one or more of the genes listed in Table 2, a primer set capable of generating an amplification product of DNA complementary to RNA encoded, in a human subject, only by the gene. In one embodiment, the set of genes comprises ASGR2 and STAB1. In another embodiment, the set of genes comprises ASGR2, C3AR1 and/or STAB1.

In another aspect, the kit further comprises a computer-readable medium having instructions stored thereon that are operable when executed by a computer for comparing the test data representing a level of RNA encoded by the gene in blood of a human test subject to positive control data representing levels of RNA encoded by the gene in blood of human control subjects having a categorized severity of heart failure, to thereby output data representing a value indicating whether the test data and the positive control data correspond to each other, wherein correspondence between the test data and the positive control data indicates that the test subject has the categorized severity of heart failure.

In another embodiment, the computer readable medium further has instructions stored thereon that are operable when executed by a computer for comparing a second positive control data representing levels of RNA encoded by the gene in blood of human control subjects having a second categorized severity of heart failure, wherein correspondence between the test data and the second positive control data indicates that the test subject has the second categorized severity of heart failure.

In yet another aspect, there is provided a kit comprising packaging and containing, for each gene of a set of one or more of the genes set forth in Table 3, a primer set capable of generating an amplification product of DNA complementary to RNA encoded, in a human subject, only by the gene. In one embodiment, the set of one or more genes comprises FAM134B, MGAT4A, ZCCHC14 and/or CD28

In another aspect, the kit further comprises a thermostable polymerase, a reverse transcriptase, deoxynucleotide triphosphates, nucleotide triphosphates and/or enzyme buffer.

In yet another aspect, the kit further comprises at least one labeled probe capable of selectively hybridizing to either a sense or an antisense strand of the amplification product.

In yet another aspect of the invention, the kit further contains a computer-readable medium of the invention.

In one aspect, the kit is identified in print in or on the packaging as being for determining severity of heart failure in a test subject, for example, a probability that a test subject has a particular heart failure classification as opposed to not having the particular heart failure classification.

In another aspect, the kit is identified in print in or on the packaging as being for monitoring the progression of heart failure in a test subject.

In a further aspect, the kit is identified in print in or on the packaging as being for classifying whether a test subject has decompensated heart failure as opposed to not having decompensated heart failure.

In yet another aspect, the kit is identified in print in or on the packaging as being for determining whether a human subject with heart failure has a prognosis of mortality as opposed to not having a prognosis of mortality.

In yet a further aspect, the kit is identified in print in or on the packaging as being for ranking a group of human test subjects based on relative risk.

In various aspects of the kits described herein, the set of genes may be any combination of two or more of the target genes, as described hereinabove and in the Examples section, below.

The disclosure also provides primer sets, isolated compositions and test systems.

Examples of a primer of the disclosure include an oligonucleotide which is capable of acting as a point of initiation of polynucleotide synthesis along a complementary strand when placed under conditions in which synthesis of a primer extension product which is complementary to a polynucleotide is catalyzed. Such conditions include the presence of four different nucleotide triphosphates or nucleoside analogs and one or more agents for polymerization such as DNA polymerase and/or reverse transcriptase, in an appropriate buffer (“buffer” includes substituents which are cofactors, or which affect pH, ionic strength, etc.), and at a suitable temperature. A primer must be sufficiently long to prime the synthesis of extension products in the presence of an agent for polymerase. A typical primer contains at least about 5 nucleotides in length of a sequence substantially complementary to the target sequence, but somewhat longer primers are preferred.

The terms “complementary” or “complement thereof”, as used herein, refer to sequences of polynucleotides which are capable of forming Watson & Crick base pairing with another specified polynucleotide throughout the entirety of the complementary region. This term is applied to pairs of polynucleotides based solely upon their sequences and does not refer to any specific conditions under which the two polynucleotides would actually bind.

A primer will always contain a sequence substantially complementary to the target sequence, that is the specific sequence to be amplified, to which it can anneal.

A primer which “selectively hybridizes” to a target polynucleotide is a primer which is capable of hybridizing only, or mostly, with a single target polynucleotide in a mixture of polynucleotides consisting of RNA of human blood, or consisting of DNA complementary to RNA of human blood.

Accordingly, there is provided an isolated composition comprising, a blood sample from a test subject and for each gene of a set of one or more genes selected from the genes listed in Table 2, one or more components selected from the group consisting of exogenous RNA encoded by the gene, cDNA complementary to the RNA, an oligonucleotide which specifically hybridizes to the cDNA or the RNA under stringent conditions, a primer set capable of generating an amplification product of the cDNA complementary to RNA, and an amplification product of the cDNA. In one embodiment, the set of genes consists of an ASGR2 gene and a STAB1 gene.

In another aspect, there is provided an isolated composition comprising, for each gene of a set of one or more genes selected from the genes listed in Table 2, a blood sample from a test subject and one or more components selected from the group consisting of exogenous RNA encoded by the gene, cDNA complementary to the RNA, an oligonucleotide which specifically hybridizes to the cDNA or the RNA under stringent conditions, a primer set capable of generating an amplification product of the cDNA complementary to RNA, and an amplification product of the cDNA. In one embodiment, the set of genes comprises or consists of an ASGR2 gene and a STAB1 gene.

In one embodiment, there is provided an isolated composition comprising a blood sample from a test subject and one or more of exogenous RNA encoded by an ASGR2 gene, a C3AR1 gene or a STAB1 gene, cDNA complementary to the RNA, an oligonucleotide which specifically hybridizes to the cDNA or the RNA under stringent conditions, a primer set capable of generating an amplification product of the cDNA complementary to RNA, and/or an amplification product of the cDNA. In another embodiment, there is provided an isolated composition comprising an isolated nucleic acid molecule of a blood sample from a test subject, wherein the nucleic acid molecule is one or more of exogenous RNA encoded by an ASGR2 gene, a C3AR1 gene or a STAB1 gene, cDNA complementary to the RNA, an oligonucleotide which specifically hybridizes to the cDNA or the RNA under stringent conditions, a primer set capable of generating an amplification product of the cDNA complementary to RNA, and/or an amplification product of the cDNA.

There is also provided an isolated composition comprising a blood sample from a test subject and one or more of exogenous RNA encoded by an FAM134B gene, a MGAT4A gene, a ZCCHC14 gene or a CD28 gene, cDNA complementary to the RNA, an oligonucleotide which specifically hybridizes to the cDNA or the RNA under stringent conditions, a primer set capable of generating an amplification product of the cDNA complementary to RNA, and/or an amplification product of the cDNA. Also provided is n isolated composition comprising an isolated nucleic acid molecule of a blood sample from a test subject, wherein the nucleic acid molecule is one or more of exogenous RNA encoded by an FAM134B gene, a MGAT4A gene, a ZCCHC14 gene or a CD28 gene, cDNA complementary to the RNA, an oligonucleotide which specifically hybridizes to the cDNA or the RNA under stringent conditions, a primer set capable of generating an amplification product of the cDNA complementary to RNA, and/or an amplification product of the cDNA.

In yet another aspect, there is provided a primer set comprising a first primer and a second primer, wherein the first primer is one of a set of primers capable of generating an amplification product of cDNA complementary to RNA encoded by a first gene, wherein the second primer is capable of generating an amplification product of cDNA complementary to RNA encoded by a second gene, and wherein the first gene and the second gene are different genes selected from the genes listed in Table 2, or composition thereof. In one embodiment, the set of genes comprises or consists of an ASGR2 gene and a STAB1 gene.

In yet another aspect, there is provided a primer set comprising a first primer, wherein the first primer is one of a set of primers capable of generating an amplification product of cDNA complementary to RNA encoded by an ASGR2 gene, and a second primer, wherein the second primer is one of a set of primers capable of generating an amplification product of cDNA complementary to RNA encoded by a C3AR1 gene, or composition thereof. Also provided is a primer set comprising a first primer, wherein the first primer is one of a set of primers capable of generating an amplification product of cDNA complementary to RNA encoded by an ASGR2 gene, and a second primer, wherein the second primer is one of a set of primers capable of generating an amplification product of cDNA complementary to RNA encoded by a STAB1 gene, or composition thereof. Further provided is a primer set comprising a first primer, wherein the first primer is one of a set of primers capable of generating an amplification product of cDNA complementary to RNA encoded by an C3AR1 gene, and a second primer, wherein the second primer is one of a set of primers capable of generating an amplification product of cDNA complementary to RNA encoded by a STAB1 gene, or composition thereof.

In a further aspect, there is provided a primer set comprising a first primer, wherein the first primer is one of a set of primers capable of generating an amplification product of cDNA complementary to RNA encoded by an FAM134B gene, and a second primer, wherein the second primer is one of a set of primers capable of generating an amplification product of cDNA complementary to RNA encoded by a MGAT4A gene, or composition thereof. Also provided is a primer set comprising a first primer, wherein the first primer is one of a set of primers capable of generating an amplification product of cDNA complementary to RNA encoded by an FAM134B gene, and a second primer, wherein the second primer is one of a set of primers capable of generating an amplification product of cDNA complementary to RNA encoded by a ZCCHC14 gene, or composition thereof. Further provided is primer set comprising a first primer, wherein the first primer is one of a set of primers capable of generating an amplification product of cDNA complementary to RNA encoded by an FAM134B gene, and a second primer, wherein the second primer is one of a set of primers capable of generating an amplification product of cDNA complementary to RNA encoded by a CD28 gene, or composition thereof. Also provided is a primer set comprising a first primer, wherein the first primer is one of a set of primers capable of generating an amplification product of cDNA complementary to RNA encoded by an MGAT4A gene, and a second primer, wherein the second primer is one of a set of primers capable of generating an amplification product of cDNA complementary to RNA encoded by a ZCCHC14 gene, or composition thereof. Further provided is a primer set comprising a first primer, wherein the first primer is one of a set of primers capable of generating an amplification product of cDNA complementary to RNA encoded by an MGAT4A gene, and a second primer, wherein the second primer is one of a set of primers capable of generating an amplification product of cDNA complementary to RNA encoded by a CD28 gene, or composition thereof. In addition, there is provided a primer set comprising a first primer, wherein the first primer is one of a set of primers capable of generating an amplification product of cDNA complementary to RNA encoded by an ZCCHC14 gene, and a second primer, wherein the second primer is one of a set of primers capable of generating an amplification product of cDNA complementary to RNA encoded by a CD28 gene, or composition thereof.

In a further aspect, there is provided a test system comprising: a) two or more blood samples wherein each blood sample is from a different test subject, and b) an isolated nucleic acid molecule of each of said blood samples, wherein said nucleic acid molecule is one or more of exogenous RNA encoded by an ASGR2, C3AR1 or STAB 1 gene, cDNA complementary to said RNA, an oligonucleotide which specifically hybridizes to said cDNA or complement thereof, or said RNA under stringent conditions, a primer set capable of generating an amplification product of said cDNA complementary to RNA, and/or an amplification product of said cDNA.

In yet another aspect, there is provided a test system comprising: (a) two or more blood samples wherein each blood sample is from a different test subject, and (b) an isolated nucleic acid molecule of each of said blood samples, wherein said nucleic acid molecule is one or more of exogenous RNA encoded by an FAM134B, MGAT4A, ZCCHC14 or CD28 gene, cDNA complementary to said RNA, an oligonucleotide which specifically hybridizes to said cDNA or complement thereof, or said RNA under stringent conditions, a primer set capable of generating an amplification product of said cDNA complementary to RNA, and/or an amplification product of said cDNA.

The above disclosure generally describes the present disclosure. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

### Results

### Subject Recruitment

A total of 87 subjects were recruited in this study: 15 were control (non-heart failure); 72 were heart failure patients. Heart failure subjects were categorized into two groups: 32 were compensated heart failure patients (NYHA I-II); 40 were de-compensated heart failure patients (NYHA III-IV). Demographic characteristics and medications of all subjects were summarized in Table 1.

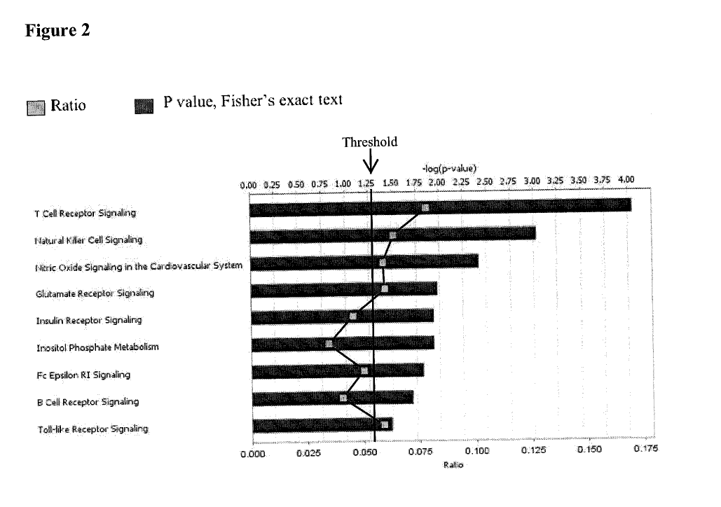

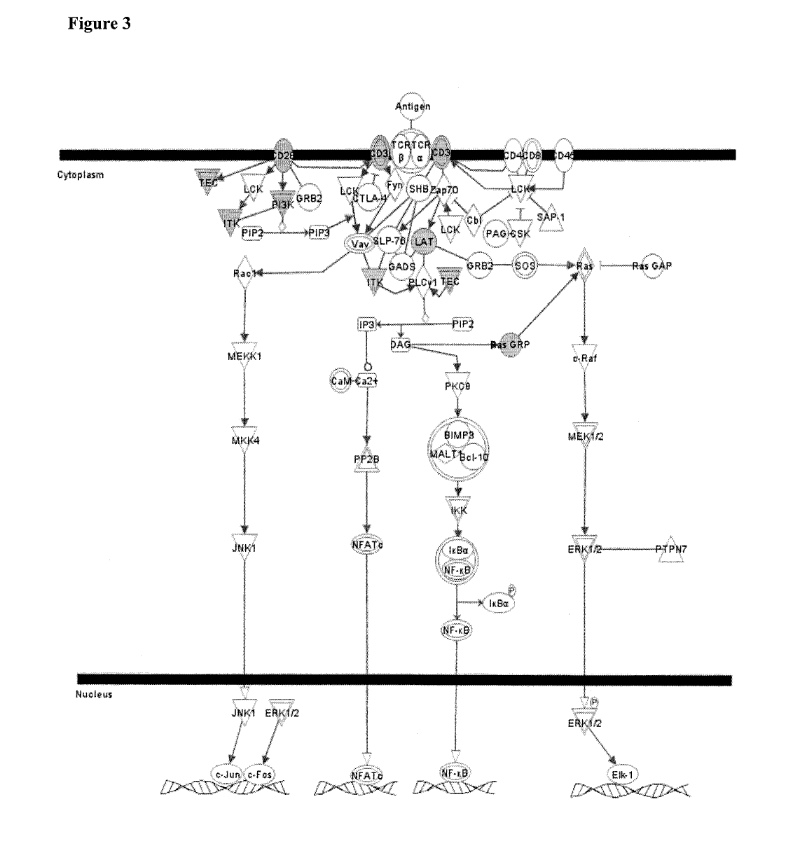

Microarray analysis identified 294 genes differentially regulated (p<0.001) in HF (FIG. 1), including the genes ASGR2, C3AR1 and STAB1 (Table 2, listed in order of increasing p-value/decreasing statistical significance). Pathway analysis revealed that genes involved in T cell receptor signalling and natural killer cell signalling were significantly (p<0.001) over-presented in HF-regulated genes (FIG. 2). HF-regulated genes in the T cell receptor signalling pathway include genes in the upstream of the pathway, such as receptors, cell surface molecules and signal transduction molecules (Table 4; FIG. 3); their expression levels decreased in HF, and the magnitude of their differential expression increased with the severity of HF (Table 4).

Analysis of Gene Expression: Affymetrix GeneChip U133Plus2.0 is a whole-genome microarray containing over 56,000 probe sets. Cross-gene error model was applied to the 87 blood expression profiles processed with GC-RMA; after removing unreliable measurements, approximately 27,000 probe sets remained for further analysis. Of these probe sets, survival analysis and subsequent multi-test correction identified the genes listed in Table 3 as significantly (q<0.2) associated with survival time.

Functional categorization of the “survival associated genes” revealed that one functional group over-presented in this group was the one involved in T-cell receptor signaling as shown in Table 4 and FIG. 3.

Certain HF-regulated genes listed in Table 2 were associated with the survival time of HF patients with a statistical significance of p<0.05 (Table 3, listed in order of increasing p-value/decreasing statistical significance); including the genes FAM134B, MGAT4A, ZCCHC14 and CD28. Below are representative equations for ranking each of a group of heart failure patients according to probability of fatal outcome.

Briefly, a person skilled in the art would be able to apply survival analysis to the genes listed in Table 3 with the Survival package in R: the expression data of each gene and the survival data is fit with a Cox proportional hazards regression model; the significance of the association between gene expression and survival time can be assessed using a logrank test; Multi-test correction is performed using the Q value (Storey and Tibshirani, 2003) package in R; a q value of 0.2 was chosen as a significance cut-off for “survival associated gene” selection. Thus, a person skilled in the art would be able to rank each of a group of test subjects according to relative risk of death using the genes listed in Table 3 by applying the general formula: relative risk=coefficient̂expression, where coefficient refers to the gene-specific coefficient value listed in Table 3, and expression refers to the log of the gene-specific RNA level in blood of the test subject, determined, e.g. as described in Materials and Methods. The symbol “̂” indicates, according to convention, that the indicated gene-specific numerical coefficient is raised to an exponent corresponding to the log of the RNA level. Such ranking has utility, for example, for prioritizing patients to be monitored and/or treated, particularly in a context of limited monitoring and/or treatment resources requiring allocation.

The below representative equations were derived using the Cox method described herein to provide the risk score for a subject.

CD28: relative risk=0.451̂Expression
FAM134B: relative risk=0.192̂Expression
MGAT4A: relative risk=0.206̂Expression
ZCCHC14: relative risk=0.440̂Expression

### Material and Methods

Subject recruitment. Heart failure subjects were identified from an outpatient clinic population or at the time of admission to hospital with primary diagnosis of HF. All patients had assessment of left ventricular function as part of routine cardiac care prior enrolment. The severity of HF was characterized using New York Heart Association (NYHA) classification. Controls were identified through the stress lab referred for atypical or non-cardiac chest pain and had no prior diagnosis of cardiac disease. Through this mechanism both the absence of significant coronary disease and normal ventricular function were confirmed by a negative stress test (stress echo and/or nuclear perfusion imaging).

Blood collection, RNA extraction and microarray hybridization. Overnight fasting blood samples were collected using a Vacutainer™ tube and stored on ice till RNA extraction. Blood samples were processed for RNA extraction within six hours after blood collection. Red blood cells were ruptured with hypotonic haemolysis buffer, followed by collection of white blood cells by centrifugation. White blood cell total RNA was extracted with Trizol® Reagent. The quality of RNA samples was assessed on an Agilent Bioanalyzer 2100 using RNA 6000 Nano Chips; the quantity of RNA was measured by UV spectrophotometry. Five microgram of total RNA of each sample was used for hybridization on a GeneChip U133Plus2.

Data analysis. Probe-level expression data were processed by GC-Robust Multichip Analysis (GC-RMA) using GeneSpring v7.3 software. Genes showing unreliable measurements, assessed by cross-gene error model, were removed from any further analysis. Differentially regulated genes by heart failure were identified by applying ANOVA to the three sample groups: control, NYHA I-II and NYHA III-IV; a p value of 0.001 was chosen as the significance cut-off. Genes with significant differential expression were subjected to cluster analysis using Spearman correlation and average linkage. Functional categorization of the HF-regulated genes were conducted using the Ingenuity Pathway Analysis software.

Survival analysis over a period of 43 months was applied to HF-regulated genes with the Survival package in R: the expression data of each gene and the survival data were fit with a Cox proportional hazards regression model; the significance of the association between gene expression and survival time was assessed using a logrank test; Multi-test correction was performed using the Q value (Storey and Tibshirani, 2003) package in R; a q value of 0.2 was chosen as a significance cut-off for “survival associate gene” selection. Differentially regulated “survival associated genes” between control and NYHA I-II, and between control and NYHA III-IV were identified by a Welch t-test; a p value of 0.05 was chosen as the significance cut-off.

Functional categorization of the “survival associated genes” was conducted using the Ingenuity Pathway Analysis software (Ingenuity Systems Inc., Redwood City, Calif.). Genes in significantly over-presented functional group(s) were subjected to cluster analysis using Pearson correlation and complete linkage. The 87 samples were re-classified based on the cluster analysis of “survival associated genes” into three groups. Survival analysis was applied to the reclassified three groups and to the original three groups based on NYHA classification (Control, NYHA I-II and NYHA III-IV); Kaplan-Meier plot was drawn using the Survival package in R; the significance was assessed using a logrank test.

### Example 2

### Classification of a Patient Suspected of Potentially Having Heart Failure as Having NYHA I-II or NYHA III/IV Stage Heart Failure

Overnight fasting blood samples are collected from a patient suspected of potentially having heart failure using a Vacutainer™ tube, and from healthy subjects and are stored on ice. The blood samples are processed for RNA extraction within six hours after blood collection. Red blood cells in the samples are ruptured with hypotonic haemolysis buffer, followed by collection of white blood cells by centrifugation. White blood cell total RNA is extracted with Trizol® Reagent. The quality of RNA samples is assessed on an Agilent Bioanalyzer 2100 using RNA 6000 Nano Chips; and the quantity of RNA is measured by UV spectrophotometry. Five micrograms of total RNA of the samples is used for hybridization on a GeneChip U133Plus2 to measure the levels of RNA encoded by the genes ASGR2 and STAB1 in the samples.

The ratio of the level of RNA encoded by ASGR2 in the sample from the patient to the average level of RNA encoded by ASGR2 in the blood samples of the healthy subjects is determined, and the ratio of the level of RNA encoded by STAB1 in the sample from the patient to the average level of RNA encoded by STAB1 in the blood samples of the healthy subjects is determined.

The patient is classified as having NYHA I/II stage heart failure if the level of RNA encoded by ASGR2 in the sample from the patient is between 1.59 to 2.45 fold, relative to the average level of RNA encoded by the gene in the blood samples of the healthy subjects, and if the level of RNA encoded by STAB1 in the sample from the patient is between 1.33 to 1.92 fold, relative to the average level of RNA encoded by the gene in the blood samples of the healthy subjects.

Alternately, the patient is classified as having NYHA III/IV stage heart failure if the level of RNA encoded by ASGR2 in the sample from the patient is greater than 2.45, relative to the average level of RNA encoded by the gene in the blood samples of the healthy subjects, and if the level of RNA encoded by STAB1 in the sample from the patient is greater than 1.92, relative to the average level of RNA encoded by the gene in the blood samples of the healthy subjects.

### Example 3

### Ranking of Patients Having Heart Failure According to Survival Time Prognosis

Overnight fasting blood samples are collected from patients diagnosed as having heart failure, using a Vacutainer™ tube and are stored on ice. The blood samples are processed for RNA extraction within six hours after blood collection. Red blood cells in the samples are ruptured with hypotonic haemolysis buffer, followed by collection of white blood cells by centrifugation. White blood cell total RNA is extracted with Trizol® Reagent. The quality of RNA samples is assessed on an Agilent Bioanalyzer 2100 using RNA 6000 Nano Chips; and the quantity of RNA is measured by UV spectrophotometry. Five micrograms of total RNA of the samples is used for hybridization on a GeneChip U133Plus2 to measure levels of RNA encoded by the gene FAM134B in the samples.

A relative risk score for risk of death for each patient is calculated according to the equation; risk score=0.192̂[level of FAM134B RNA in sample]; and the patients are ranked according to survival time prognosis as a function of risk score, where the higher the risk score, the worse the survival time prognosis.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. In addition all sequences identified herein by accession number for example in Tables herein, are also specifically incorporated by reference.

TABLE 1

| | Control | NYHA I-II | NYHA III-IV |
|---|---|---|---|
| N | 15 | 32 | 40 |
| Age: mean (range) | 58.0 | 60.2 (28.2-87.7) | 66.9 (46.9-85.4) |
| | (33.6-81.1) | | |
| Sex: Male/Female | 7-Aug | 21-Nov | 28-Dec |
| LVEFpc: mean | 63 (55-77) | 27 (10-64) | 22 (10-60) |
| (range) | | | |
| Ischemic | 0% | 43.80% | 62.50% |
| Cardiomyopathy | | | |
| BNP: mean (range) | — | 1249 (80-2230) | 1251 (182-4890) |
| Diabetes Mellitus | 20.00% | 21.90% | 52.50% |
| Hypertension | 60.00% | 71.90% | 77.50% |
| Renal Insufficiency | 0.00% | 9.40% | 37.50% |
| Atrial Fibrillation | 0.00% | 28.10% | 52.50% |
| Loop Diuretic | 20.00% | 34.40% | 80.00% |
| β blocker | 40.00% | 81.30% | 82.50% |
| ACE-I/ARB | 26.70% | 87.50% | 77.50% |

TABLE 2
Severity genes - note that genes are listed in order of decreasing
statistical significance/preference.

| | | | | | Fold-change | |
|---|---|---|---|---|---|---|
| | | Gene | | | expression | Fold-change expression |
| Probe set | RefSeq | symbol | Gene description | ANOVA p | (NYHA I-II/Control) | (NYHA III-IV/Control) |
| 206130_s_at | NM_001181 | ASGR2 | asialoglycoprotein | 3.49E−07 | 1.59 | 2.45 |
| | | | receptor 2 | | | |
| 209906_at | NM_004054 | C3AR1 | complement | 8.50E−07 | 1.05 | 1.94 |
| | | | component 3a receptor 1 | | | |
| 204150_at | NM_015136 | STAB1 | stabilin 1 | 1.55E−06 | 1.33 | 1.92 |
| 225864_at | NM_174911 | FAM84B | family with sequence | 2.72E−06 | 0.82 | 0.56 |
| | | | similarity 84, member B | | | |
| 212331_at | NM_005611 | RBL2 | retinoblastoma-like 2 | 4.46E−06 | 1.00 | 0.85 |
| | | | (p130) | | | |
| 232633_at | NM_021141 | XRCC5 | X-ray repair | 4.94E−06 | 0.90 | 0.80 |
| | | | complementing | | | |
| | | | defective repair in | | | |
| | | | Chinese hamster cells | | | |
| | | | 5 (double-strand-break | | | |
| | | | rejoining; Ku | | | |
| | | | autoantigen, 80 kDa) | | | |
| 222820_at | NM_018996 | TNRC6C | trinucleotide repeat | 5.98E−06 | 0.96 | 0.71 |
| | | | containing 6C | | | |
| 222585_x_at | NM_016618 | KRCC1 | lysine-rich coiled-coil 1 | 6.05E−06 | 1.04 | 1.20 |
| 226148_at | NM_014155 | ZBTB44 | zinc finger and BTB | 1.18E−05 | 0.91 | 0.80 |
| | | | domain containing 44 | | | |
| 218306_s_at | NM_003922 | HERC1 | hect (homologous to | 1.88E−05 | 0.91 | 0.86 |
| | | | the E6-AP (UBE3A) | | | |
| | | | carboxyl terminus) | | | |
| | | | domain and RCC1 | | | |
| | | | (CHC1)-like domain | | | |
| | | | (RLD) 1 | | | |
| 228047_at | NR_002581 | SNORA72 | small nucleolar RNA, | 2.06E−05 | 0.90 | 0.76 |
| | | | H/ACA box 72 | | | |
| 217388_s_at | NM_001032998 | KYNU | kynureninase (L- | 2.19E−05 | 1.00 | 1.58 |
| | | | kynurenine hydrolase) | | | |
| 210102_at | NM_014622 | LOH11CR2A | loss of heterozygosity, | 2.80E−05 | 1.19 | 1.68 |
| | | | 11, chromosomal | | | |
| | | | region 2, gene A | | | |
| 228624_at | NM_018342 | TMEM144 | transmembrane protein | 2.84E−05 | 1.34 | 1.98 |
| | | | 144 | | | |
| 202664_at | NM_001077269 | WIPF1 | WAS/WASL interacting | 3.13E−05 | 0.94 | 0.86 |
| | | | protein family, member 1 | | | |
| 202165_at | NM_006241 | PPP1R2 | protein phosphatase 1, | 3.61E−05 | 0.92 | 0.82 |
| | | | regulatory (inhibitor) | | | |
| | | | subunit 2 | | | |
| 228106_at | NM_017741 | C4orf30 | chromosome 4 open | 4.09E−05 | 1.03 | 0.73 |
| | | | reading frame 30 | | | |
| 235048_at | NM_015566 | KIAA0888 | KIAA0888 protein | 4.58E−05 | 0.85 | 0.57 |
| 226529_at | NM_018374 | TMEM106B | transmembrane protein | 4.66E−05 | 0.92 | 0.80 |
| | | | 106B | | | |
| 205259_at | NM_000901 | NR3C2 | nuclear receptor | 4.73E−05 | 0.82 | 0.50 |
| | | | subfamily 3, group C, | | | |
| | | | member 2 | | | |
| 226158_at | NM_017644 | KLHL24 | kelch-like 24 | 4.90E−05 | 0.95 | 0.77 |
| | | | (Drosophila) | | | |
| 225724_at | — | FLJ31306 | hypothetical protein | 5.17E−05 | 0.96 | 0.83 |
| | | | FLJ31306 | | | |
| 205698_s_at | NM_002758 | MAP2K6 | mitogen-activated | 5.27E−05 | 0.81 | 1.39 |
| | | | protein kinase kinase 6 | | | |
| 203408_s_at | NM_002971 | SATB1 | SATB homeobox 1 | 5.42E−05 | 0.95 | 0.74 |
| 206857_s_at | NM_004116 | FKBP1B | FK506 binding protein | 5.48E−05 | 1.11 | 1.78 |
| | | | 1B, 12.6 kDa | | | |
| 211571_s_at | NM_004385 | VCAN | versican | 5.75E−05 | 1.24 | 1.70 |
| 213908_at | NR_003521 | WHDC1L1 | WAS protein homology | 5.82E−05 | 0.89 | 0.67 |
| | | | region 2 domain | | | |
| | | | containing 1-like 1 | | | |
| 204642_at | NM_001400 | EDG1 | endothelial | 6.34E−05 | 0.95 | 0.67 |
| | | | differentiation, | | | |
| | | | sphingolipid G-protein- | | | |
| | | | coupled receptor, 1 | | | |
| 208771_s_at | NM_000895 | LTA4H | leukotriene A4 | 7.10E−05 | 1.08 | 1.45 |
| | | | hydrolase | | | |
| 218411_s_at | NM_016586 | MBIP | MAP3K12 binding | 7.64E−05 | 1.05 | 0.79 |
| | | | inhibitory protein 1 | | | |
| 230490_x_at | NM_012425 | RSU1 | Ras suppressor protein 1 | 7.67E−05 | 1.12 | 0.85 |
| 202970_at | NM_003583 | DYRK2 | dual-specificity | 7.76E−05 | 1.00 | 0.73 |
| | | | tyrosine-(Y)- | | | |
| | | | phosphorylation | | | |
| | | | regulated kinase 2 | | | |
| 212538_at | NM_015296 | DOCK9 | dedicator of cytokinesis 9 | 7.93E−05 | 0.93 | 0.64 |
| 226682_at | — | LOC283666 | hypothetical protein | 8.06E−05 | 0.98 | 0.64 |
| | | | LOC283666 | | | |
| 209884_s_at | NM_003615 | SLC4A7 | solute carrier family 4, | 8.62E−05 | 0.99 | 0.75 |
| | | | sodium bicarbonate | | | |
| | | | cotransporter, member 7 | | | |
| 228577_x_at | NM_001007022 | ODF2L | outer dense fiber of | 9.04E−05 | 0.93 | 0.76 |
| | | | sperm tails 2-like | | | |
| 227626_at | NM_133367 | PAQR8 | progestin and adipoQ | 9.07E−05 | 0.88 | 0.68 |
| | | | receptor family | | | |
| | | | member VIII | | | |
| 224918_x_at | NM_020300 | MGST1 | microsomal glutathione | 9.11E−05 | 1.28 | 1.68 |
| | | | S-transferase 1 | | | |
| 227067_x_at | NM_203458 | NOTCH2NL | Notch homolog 2 | 9.51E−05 | 1.04 | 1.59 |
| | | | (Drosophila) N-terminal | | | |
| | | | like | | | |
| 1559097_at | — | C14orf64 | chromosome 14 open | 9.75E−05 | 0.94 | 0.59 |
| | | | reading frame 64 | | | |
| 212672_at | NM_000051 | ATM | ataxia telangiectasia | 9.85E−05 | 1.00 | 0.75 |
| | | | mutated | | | |
| 227639_at | NM_005482 | PIGK | phosphatidylinositol | 0.0001 | 0.86 | 0.71 |
| | | | glycan anchor | | | |
| | | | biosynthesis, class K | | | |
| 204165_at | NM_001024934 | WASF1 | WAS protein family, | 0.000101 | 1.23 | 1.74 |
| | | | member 1 | | | |
| 226327_at | NM_014910 | ZNF507 | zinc finger protein 507 | 0.000101 | 0.97 | 0.80 |
| 211985_s_at | NM_006888 | CALM1 | calmodulin 1 | 0.000105 | 0.98 | 0.84 |
| | | | (phosphorylase kinase, | | | |
| | | | delta) | | | |
| 203556_at | NM_014943 | ZHX2 | zinc fingers and | 0.000107 | 0.83 | 0.79 |
| | | | homeoboxes 2 | | | |
| 205434_s_at | NM_001012987 | AAK1 | AP2 associated kinase 1 | 0.00011 | 0.94 | 0.73 |
| 228423_at | NM_001039580 | MAP9 | microtubule-associated | 0.000112 | 0.91 | 0.61 |
| | | | protein 9 | | | |
| 242945_at | NM_017565 | FAM20A | family with sequence | 0.000117 | 1.11 | 1.96 |
| | | | similarity 20, member A | | | |
| 225117_at | NM_015443 | KIAA1267 | KIAA1267 | 0.000122 | 0.92 | 0.86 |
| 200686_s_at | NM_004768 | SFRS11 | splicing factor, | 0.000123 | 1.06 | 0.85 |
| | | | arginine/serine-rich 11 | | | |
| 212609_s_at | NM_005465 | AKT3 | V-akt murine thymoma | 0.000126 | 0.92 | 0.73 |
| | | | viral oncogene | | | |
| | | | homolog 3 (protein | | | |
| | | | kinase B, gamma) | | | |
| 230529_at | NM_016217 | HECA | headcase homolog | 0.000129 | 0.86 | 0.81 |
| | | | (Drosophila) | | | |
| 210156_s_at | NM_005389 | PCMT1 | protein-L-isoaspartate | 0.000131 | 0.96 | 1.17 |
| | | | (D-aspartate) O- | | | |
| | | | methyltransferase | | | |
| 219607_s_at | NM_024021 | MS4A4A | membrane-spanning 4- | 0.000134 | 1.14 | 2.80 |
| | | | domains, subfamily A, | | | |
| | | | member 4 | | | |
| 200663_at | NM_001040034 | CD63 | CD63 molecule | 0.00014 | 0.99 | 1.20 |
| 202723_s_at | NM_002015 | FOXO1 | forkhead box O1 | 0.000142 | 0.84 | 0.81 |
| 204075_s_at | NM_014704 | KIAA0562 | KIAA0562 | 0.000143 | 1.04 | 0.82 |
| 201656_at | NM_000210 | ITGA6 | integrin, alpha 6 | 0.000145 | 0.96 | 0.64 |
| 223993_s_at | NM_014184 | CNIH4 | cornichon homolog 4 | 0.000146 | 0.85 | 1.23 |
| | | | (Drosophila) | | | |
| 204484_at | NM_002646 | PIK3C2B | phosphoinositide-3- | 0.000148 | 0.87 | 0.63 |
| | | | kinase, class 2, beta | | | |
| | | | polypeptide | | | |
| 213224_s_at | — | LOC92482 | hypothetical protein | 0.000148 | 0.89 | 0.83 |
| | | | LOC92482 | | | |
| 212205_at | NM_012412 | H2AFV | H2A histone family, | 0.000149 | 0.90 | 0.79 |
| | | | member V | | | |
| 219387_at | NM_018084 | CCDC88A | coiled-coil domain | 0.000153 | 1.17 | 1.90 |
| | | | containing 88A | | | |
| 209604_s_at | NM_001002295 | GATA3 | GATA binding protein 3 | 0.000154 | 1.02 | 0.66 |
| 226247_at | NM_001001974 | PLEKHA1 | pleckstrin homology | 0.000159 | 0.98 | 0.69 |
| | | | domain containing, | | | |
| | | | family A | | | |
| | | | (phosphoinositide | | | |
| | | | binding specific) | | | |
| | | | member 1 | | | |
| 201850_at | NM_001747 | CAPG | capping protein (actin | 0.000159 | 1.00 | 1.55 |
| | | | filament), gelsolin-like | | | |
| 225191_at | NM_001280 | CIRBP | cold inducible RNA | 0.000162 | 0.89 | 0.84 |
| | | | binding protein | | | |
| 201557_at | NM_014232 | VAMP2 | vesicle-associated | 0.000163 | 1.01 | 0.81 |
| | | | membrane protein 2 | | | |
| | | | (synaptobrevin 2) | | | |
| 222981_s_at | NM_016131 | RAB10 | RAB10, member RAS | 0.000168 | 0.99 | 1.27 |
| | | | oncogene family | | | |
| 227448_at | NM_018011 | FLJ10154 | hypothetical protein | 0.00017 | 0.94 | 0.78 |
| | | | FLJ10154 | | | |
| 202821_s_at | NM_005578 | LPP | LIM domain containing | 0.000171 | 0.90 | 1.20 |
| | | | preferred translocation | | | |
| | | | partner in lipoma | | | |
| 212455_at | NM_001031732 | YTHDC1 | YTH domain containing 1 | 0.000182 | 0.96 | 0.92 |
| 1555037_a_at | NM_005896 | IDH1 | isocitrate | 0.000191 | 1.13 | 1.39 |
| | | | dehydrogenase 1 | | | |
| | | | (NADP+), soluble | | | |
| 204773_at | NM_004512 | IL11RA | interleukin 11 receptor, | 0.000192 | 1.11 | 0.74 |
| | | | alpha | | | |
| 228446_at | NM_001017969 | KIAA2026 | KIAA2026 | 0.000192 | 0.94 | 0.85 |
| 205005_s_at | NM_004808 | NMT2 | N-myristoyltransferase 2 | 0.000199 | 0.94 | 0.55 |
| 230078_at | NM_016340 | RAPGEF6 | Rap guanine | 0.000201 | 0.95 | 0.82 |
| | | | nucleotide exchange | | | |
| | | | factor (GEF) 6 | | | |
| 201007_at | NM_000183 | HADHB | hydroxyacyl-Coenzyme | 0.000203 | 0.98 | 1.13 |
| | | | A dehydrogenase/3- | | | |
| | | | ketoacyl-Coenzyme A | | | |
| | | | thiolase/enoyl- | | | |
| | | | Coenzyme A hydratase | | | |
| | | | (trifunctional protein), | | | |
| | | | beta subunit | | | |
| 221493_at | NM_003309 | TSPYL1 | TSPY-like 1 | 0.000212 | 0.93 | 0.80 |
| 221905_at | NM_001042355 | CYLD | cylindromatosis (turban | 0.000214 | 1.03 | 0.79 |
| | | | tumor syndrome) | | | |
| 1556402_at | — | FLJ46446 | Hypothetical gene | 0.000217 | 0.86 | 0.56 |
| | | | supported by | | | |
| | | | AK128305 | | | |
| 214049_x_at | NM_006137 | CD7 | CD7 molecule | 0.000218 | 0.88 | 0.71 |
| 214442_s_at | NM_004671 | PIAS2 | protein inhibitor of | 0.00022 | 0.79 | 1.23 |
| | | | activated STAT, 2 | | | |
| 222435_s_at | NM_016021 | UBE2J1 | ubiquitin-conjugating | 0.00022 | 1.03 | 1.51 |
| | | | enzyme E2, J1 (UBC6 | | | |
| | | | homolog, yeast) | | | |
| 220034_at | NM_007199 | IRAK3 | interleukin-1 receptor- | 0.00022 | 0.66 | 1.13 |
| | | | associated kinase 3 | | | |
| 231817_at | NM_019050 | USP53 | ubiquitin specific | 0.000224 | 0.96 | 0.65 |
| | | | peptidase 53 | | | |
| 212981_s_at | NM_014719 | FAM115A | family with sequence | 0.000225 | 0.93 | 0.67 |
| | | | similarity 115, member A | | | |
| 212655_at | NM_015144 | ZCCHC14 | zinc finger, CCHC | 0.000231 | 0.89 | 0.73 |
| | | | domain containing 14 | | | |
| 202419_at | NM_002035 | FVT1 | follicular lymphoma | 0.000231 | 1.02 | 0.80 |
| | | | variant translocation 1 | | | |
| 208896_at | NM_006773 | DDX18 | DEAD (Asp-Glu-Ala- | 0.000232 | 1.10 | 0.82 |
| | | | Asp) box polypeptide | | | |
| | | | 18 | | | |
| 226581_at | NM_022340 | ZFYVE20 | zinc finger, FYVE | 0.000234 | 0.94 | 0.88 |
| | | | domain containing 20 | | | |
| 224833_at | NM_005238 | ETS1 | v-ets erythroblastosis | 0.000236 | 0.96 | 0.70 |
| | | | virus E26 oncogene | | | |
| | | | homolog 1 (avian) | | | |
| 224698_at | NM_020728 | FAM62B | family with sequence | 0.000243 | 1.01 | 0.72 |
| | | | similarity 62 (C2 | | | |
| | | | domain containing) | | | |
| | | | member B | | | |
| 213034_at | NM_025164 | KIAA0999 | KIAA0999 protein | 0.000243 | 0.84 | 0.85 |
| 212343_at | NM_173834 | YIPF6 | Yip1 domain family, | 0.000247 | 1.00 | 0.86 |
| | | | member 6 | | | |
| 218499_at | NM_001042452 | RP6- | serine/threonine | 0.000248 | 0.91 | 0.82 |
| | | 213H19.1 | protein kinase MST4 | | | |
| 211946_s_at | NM_015172 | BAT2D1 | BAT2 domain | 0.000255 | 0.96 | 0.87 |
| | | | containing 1 | | | |
| 227988_s_at | NM_001018037 | VPS13A | vacuolar protein sorting | 0.000255 | 1.08 | 0.71 |
| | | | 13 homolog A (S. cerevisiae) | | | |
| 222895_s_at | NM_022898 | BCL11B | B-cell CLL/lymphoma | 0.000257 | 0.89 | 0.59 |
| | | | 11B (zinc finger | | | |
| | | | protein) | | | |
| 238614_x_at | NM_025189 | ZNF430 | zinc finger protein 430 | 0.000279 | 1.06 | 0.86 |
| 228065_at | NM_182557 | BCL9L | B-cell CLL/lymphoma | 0.00028 | 0.92 | 0.71 |
| | | | 9-like | | | |
| 225026_at | NM_032221 | CHD6 | chromodomain | 0.000282 | 1.03 | 0.79 |
| | | | helicase DNA binding | | | |
| | | | protein 6 | | | |
| 227900_at | NM_170662 | CBLB | Cas-Br-M (murine) | 0.000283 | 0.90 | 0.65 |
| | | | ecotropic retroviral | | | |
| | | | transforming sequence b | | | |
| 227119_at | NM_144571 | CNOT6L | CCR4-NOT | 0.000283 | 0.87 | 0.77 |
| | | | transcription complex, | | | |
| | | | subunit 6-like | | | |
| 206111_at | NM_002934 | RNASE2 | ribonuclease, RNase A | 0.000283 | 0.93 | 1.47 |
| | | | family, 2 (liver, | | | |
| | | | eosinophil-derived | | | |
| | | | neurotoxin) | | | |
| 222279_at | NM_001003807 | RP3- | hypothetical protein | 0.000287 | 0.99 | 0.74 |
| | | 377H14.5 | FLJ35429 | | | |
| 214470_at | NM_002258 | KLRB1 | killer cell lectin-like | 0.00029 | 0.95 | 0.66 |
| | | | receptor subfamily B, | | | |
| | | | member 1 | | | |
| 209674_at | NM_004075 | CRY1 | cryptochrome 1 | 0.000292 | 0.92 | 0.72 |
| | | | (photolyase-like) | | | |
| 214582_at | NM_000922 | PDE3B | phosphodiesterase 3B, | 0.000305 | 0.80 | 0.72 |
| | | | cGMP-inhibited | | | |
| 212660_at | NM_015288 | PHF15 | PHD finger protein 15 | 0.000306 | 0.86 | 0.78 |
| 228549_at | NM_014698 | TMEM63A | Transmembrane | 0.000314 | 0.90 | 0.72 |
| | | | protein 63A | | | |
| 226479_at | NM_152903 | KBTBD6 | kelch repeat and BTB | 0.000315 | 0.84 | 0.61 |
| | | | (POZ) domain | | | |
| | | | containing 6 | | | |
| 226753_at | NM_144664 | FAM76B | family with sequence | 0.000319 | 0.91 | 0.80 |
| | | | similarity 76, member B | | | |
| 206545_at | NM_006139 | CD28 | CD28 molecule | 0.000321 | 1.01 | 0.58 |
| 224968_at | NM_080667 | CCDC104 | coiled-coil domain | 0.000322 | 0.99 | 0.71 |
| | | | containing 104 | | | |
| 226181_at | NM_016262 | TUBE1 | tubulin, epsilon 1 | 0.000327 | 1.03 | 0.67 |
| 204203_at | NM_001806 | CEBPG | CCAAT/enhancer | 0.000339 | 1.06 | 1.40 |
| | | | binding protein | | | |
| | | | (C/EBP), gamma | | | |
| 212675_s_at | NM_015147 | CEP68 | centrosomal protein | 0.000342 | 0.98 | 0.75 |
| | | | 68 kDa | | | |
| 212259_s_at | NM_020524 | PBXIP1 | pre-B-cell leukemia | 0.000344 | 0.77 | 0.76 |
| | | | homeobox interacting | | | |
| | | | protein 1 | | | |
| 219316_s_at | NM_017791 | FLVCR2 | feline leukemia virus | 0.000344 | 1.15 | 1.75 |
| | | | subgroup C cellular | | | |
| | | | receptor family, | | | |
| | | | member 2 | | | |
| 219378_at | NM_001110798 | NARG1L | NMDA receptor | 0.000345 | 0.96 | 0.82 |
| | | | regulated 1-like | | | |
| 209368_at | NM_001979 | EPHX2 | epoxide hydrolase 2, | 0.00035 | 0.83 | 0.65 |
| | | | cytoplasmic | | | |
| 206237_s_at | NM_004495 | NRG1 | neuregulin 1 | 0.000353 | 1.20 | 1.71 |
| 226030_at | NM_001609 | ACADSB | acyl-Coenzyme A | 0.000356 | 0.98 | 0.76 |
| | | | dehydrogenase, | | | |
| | | | short/branched chain | | | |
| 228853_at | XM_001125680 | LOC730432 | similar to | 0.000359 | 0.97 | 0.79 |
| | | | serine/threonine/tyrosine | | | |
| | | | interacting protein | | | |
| 201560_at | NM_013943 | CLIC4 | chloride intracellular | 0.000359 | 1.07 | 1.51 |
| | | | channel 4 | | | |
| 224739_at | NM_001001852 | PIM3 | pim-3 oncogene | 0.000368 | 1.06 | 1.25 |
| 1553132_a_at | NM_152332 | TC2N | tandem C2 domains, | 0.00037 | 1.09 | 0.66 |
| | | | nuclear | | | |
| 1552426_a_at | NM_025141 | TM2D3 | TM2 domain containing 3 | 0.00037 | 0.98 | 0.86 |
| 212033_at | NM_021239 | RBM25 | RNA binding motif | 0.000372 | 1.05 | 0.93 |
| | | | protein 25 | | | |
| 207231_at | NM_014648 | DZIP3 | zinc finger DAZ | 0.000374 | 1.05 | 0.74 |
| | | | interacting protein 3 | | | |
| 237033_at | NM_001042693 | MGC52498 | hypothetical protein | 0.000378 | 0.93 | 0.70 |
| | | | MGC52498 | | | |
| 235125_x_at | NM_198549 | FAM73A | family with sequence | 0.000379 | 1.07 | 0.80 |
| | | | similarity 73, member A | | | |
| 227984_at | XM_944170 | LOC650392 | Hypothetical protein | 0.000384 | 1.00 | 0.66 |
| | | | LOC650392 | | | |
| 218473_s_at | NM_024656 | GLT25D1 | glycosyltransferase 25 | 0.000391 | 1.04 | 1.17 |
| | | | domain containing 1 | | | |
| 228282_at | NM_152778 | MFSD8 | Major facilitator | 0.000391 | 1.02 | 0.84 |
| | | | superfamily domain | | | |
| | | | containing 8 | | | |
| 243492_at | NM_053055 | THEM4 | Thioesterase | 0.000392 | 0.88 | 0.66 |
| | | | superfamily member 4 | | | |
| 206761_at | NM_005816 | CD96 | CD96 molecule | 0.000401 | 0.99 | 0.68 |
| 223592_s_at | NM_032322 | RNF135 | ring finger protein 135 | 0.000402 | 1.01 | 1.26 |
| 218723_s_at | NM_014059 | C13orf15 | chromosome 13 open | 0.000405 | 0.88 | 0.58 |
| | | | reading frame 15 | | | |
| 214195_at | NM_000391 | TPP1 | tripeptidyl peptidase I | 0.000412 | 1.04 | 1.10 |
| 202436_s_at | NM_000104 | CYP1B1 | cytochrome P450, | 0.000413 | 1.15 | 1.79 |
| | | | family 1, subfamily B, | | | |
| | | | polypeptide 1 | | | |
| 223092_at | NM_054027 | ANKH | ankylosis, progressive | 0.000422 | 0.95 | 0.70 |
| | | | homolog (mouse) | | | |
| 221036_s_at | NM_031301 | APH1B | anterior pharynx | 0.000424 | 0.85 | 1.11 |
| | | | defective 1 homolog B | | | |
| | | | (C. elegans) | | | |
| 1553974_at | NM_173793 | LOC128977 | hypothetical protein | 0.000427 | 0.97 | 0.85 |
| | | | LOC128977 | | | |
| 231124_x_at | NM_001033667 | LY9 | lymphocyte antigen 9 | 0.00043 | 0.93 | 0.66 |
| 1556743_at | NM_018293 | ZNF654 | zinc finger protein 654 | 0.00044 | 0.92 | 0.81 |
| 209798_at | NM_002519 | NPAT | nuclear protein, ataxia- | 0.000444 | 0.93 | 0.79 |
| | | | telangiectasia locus | | | |
| 203234_at | NM_003364 | UPP1 | uridine phosphorylase 1 | 0.000445 | 0.92 | 1.37 |
| 205936_s_at | NM_002115 | HK3 | hexokinase 3 (white | 0.000446 | 1.01 | 1.54 |
| | | | cell) | | | |
| 232914_s_at | NM_032379 | SYTL2 | synaptotagmin-like 2 | 0.000447 | 0.83 | 0.57 |
| 203939_at | NM_002526 | NT5E | 5′-nucleotidase, ecto | 0.000457 | 0.77 | 0.62 |
| | | | (CD73) | | | |
| 201666_at | NM_003254 | TIMP1 | TIMP metallopeptidase | 0.000457 | 1.06 | 1.40 |
| | | | inhibitor 1 | | | |
| 202880_s_at | NM_004762 | PSCD1 | pleckstrin homology, | 0.000458 | 0.95 | 0.89 |
| | | | Sec7 and coiled-coil | | | |
| | | | domains 1(cytohesin 1) | | | |
| 201677_at | NM_001006109 | C3orf37 | Chromosome 3 open | 0.00046 | 0.90 | 0.77 |
| | | | reading frame 37 | | | |
| 202704_at | NM_005749 | TOB1 | transducer of ERBB2, 1 | 0.000461 | 0.91 | 0.80 |
| 228760_at | NM_032102 | SFRS2B | splicing factor, | 0.000463 | 0.97 | 0.71 |
| | | | arginine/serine-rich 2B | | | |
| 220099_s_at | NM_016019 | LUC7L2 | LUC7-like 2 (S. cerevisiae) | 0.000466 | 0.99 | 0.84 |
| 226680_at | NM_022466 | IKZF5 | IKAROS family zinc | 0.000474 | 0.91 | 0.81 |
| | | | finger 5 (Pegasus) | | | |
| 224737_x_at | NM_018237 | CCAR1 | cell division cycle and | 0.000479 | 1.14 | 0.89 |
| | | | apoptosis regulator 1 | | | |
| 221221_s_at | NM_017415 | KLHL3 | kelch-like 3 | 0.000489 | 0.89 | 0.58 |
| | | | (Drosophila) | | | |
| 209657_s_at | NM_004506 | HSF2 | heat shock | 0.00049 | 0.88 | 0.72 |
| | | | transcription factor 2 | | | |
| 200965_s_at | NM_001003407 | ABLIM1 | actin binding LIM | 0.000495 | 0.90 | 0.61 |
| | | | protein 1 | | | |
| 209124_at | NM_002468 | MYD88 | myeloid differentiation | 0.00051 | 1.01 | 1.16 |
| | | | primary response gene | | | |
| | | | (88) | | | |
| 208659_at | NM_001288 | CLIC1 | chloride intracellular | 0.00051 | 0.97 | 1.12 |
| | | | channel 1 | | | |
| 207606_s_at | NM_018287 | ARHGAP12 | Rho GTPase activating | 0.000511 | 0.90 | 0.82 |
| | | | protein 12 | | | |
| 211336_x_at | NM_001081637 | LILRB1 | leukocyte | 0.000513 | 1.08 | 1.32 |
| | | | immunoglobulin-like | | | |
| | | | receptor, subfamily B | | | |
| | | | (with TM and ITIM | | | |
| | | | domains), member 1 | | | |
| 228904_at | NM_002146 | HOXB3 | homeobox B3 | 0.000516 | 1.05 | 0.78 |
| 232262_at | NM_004278 | PIGL | phosphatidylinositol | 0.000517 | 1.06 | 0.83 |
| | | | glycan anchor | | | |
| | | | biosynthesis, class L | | | |
| 223625_at | NM_032581 | FAM126A | family with sequence | 0.000522 | 1.16 | 1.41 |
| | | | similarity 126, member A | | | |
| 211282_x_at | NM_001039664 | TNFRSF25 | tumor necrosis factor | 0.000525 | 0.91 | 0.62 |
| | | | receptor superfamily, | | | |
| | | | member 25 | | | |
| 218454_at | NM_024829 | FLJ22662 | hypothetical protein | 0.000525 | 1.03 | 1.32 |
| | | | FLJ22662 | | | |
| 206896_s_at | NM_052847 | GNG7 | guanine nucleotide | 0.000531 | 0.67 | 0.72 |
| | | | binding protein (G | | | |
| | | | protein), gamma 7 | | | |
| 217118_s_at | NM_001009880 | C22orf9 | chromosome 22 open | 0.000532 | 1.10 | 1.44 |
| | | | reading frame 9 | | | |
| 225619_at | NM_001040153 | SLAIN1 | SLAIN motif family, | 0.000534 | 0.95 | 0.63 |
| | | | member 1 | | | |
| 203450_at | NM_001002880 | CBY1 | chibby homolog 1 | 0.000536 | 0.95 | 0.77 |
| | | | (Drosophila) | | | |
| 236436_at | NM_001077241 | SLC25A45 | solute carrier family 25, | 0.000536 | 0.99 | 0.83 |
| | | | member 45 | | | |
| 209734_at | NM_005337 | NCKAP1L | NCK-associated | 0.000546 | 1.01 | 1.29 |
| | | | protein 1-like | | | |
| 208807_s_at | NM_001005271 | CHD3 | chromodomain | 0.000553 | 1.00 | 0.82 |
| | | | helicase DNA binding | | | |
| | | | protein 3 | | | |
| 228026_at | NM_001102396 | SIKE | suppressor of IKK | 0.000555 | 0.99 | 0.80 |
| | | | epsilon | | | |
| 201675_at | NM_003488 | AKAP1 | A kinase (PRKA) | 0.000561 | 1.11 | 0.86 |
| | | | anchor protein 1 | | | |
| 47560_at | NM_001008701 | LPHN1 | latrophilin 1 | 0.000563 | 1.03 | 0.78 |
| 222164_at | NM_015850 | FGFR1 | fibroblast growth factor | 0.000566 | 1.11 | 0.84 |
| | | | receptor 1 (fms-related | | | |
| | | | tyrosine kinase 2, | | | |
| | | | Pfeiffer syndrome) | | | |
| 210031_at | NM_000734 | CD247 | CD247 molecule | 0.000573 | 0.88 | 0.68 |
| 205603_s_at | NM_006729 | DIAPH2 | diaphanous homolog 2 | 0.000575 | 1.07 | 1.33 |
| | | | (Drosophila) | | | |
| 212593_s_at | NM_014456 | PDCD4 | programmed cell death | 0.000576 | 0.90 | 0.80 |
| | | | 4 (neoplastic | | | |
| | | | transformation | | | |
| | | | inhibitor) | | | |
| 228950_s_at | NM_001002292 | GPR177 | G protein-coupled | 0.000577 | 0.62 | 0.73 |
| | | | receptor 177 | | | |
| 209389_x_at | NM_001079862 | DBI | diazepam binding | 0.000603 | 1.07 | 1.31 |
| | | | inhibitor (GABA | | | |
| | | | receptor modulator, | | | |
| | | | acyl-Coenzyme A | | | |
| | | | binding protein) | | | |
| 212658_at | NM_005779 | LHFPL2 | lipoma HMGIC fusion | 0.000608 | 0.77 | 1.22 |
| | | | partner-like 2 | | | |
| 206770_s_at | NM_012243 | SLC35A3 | solute carrier family 35 | 0.000614 | 1.11 | 0.87 |
| | | | (UDP-N- | | | |
| | | | acetylglucosamine | | | |
| | | | (UDP-GlcNAc) | | | |
| | | | transporter), member | | | |
| | | | A3 | | | |
| 1566448_at | NM_006725 | CD6 | CD6 molecule | 0.000616 | 0.96 | 0.73 |
| 219298_at | NM_024693 | ECHDC3 | enoyl Coenzyme A | 0.000622 | 0.50 | 0.89 |
| | | | hydratase domain | | | |
| | | | containing 3 | | | |
| 201536_at | NM_004090 | DUSP3 | dual specificity | 0.000623 | 1.12 | 1.48 |
| | | | phosphatase 3 | | | |
| | | | (vaccinia virus | | | |
| | | | phosphatase VH1- | | | |
| | | | related) | | | |
| 213926_s_at | NM_004504 | HRB | HIV-1 Rev binding | 0.000625 | 0.76 | 1.22 |
| | | | protein | | | |
| 227093_at | NM_025090 | USP36 | Ubiquitin specific | 0.000626 | 1.06 | 0.83 |
| | | | peptidase 36 | | | |
| 219351_at | NM_001011658 | TRAPPC2 | trafficking protein | 0.00063 | 0.95 | 0.83 |
| | | | particle complex 2 | | | |
| 204040_at | NM_014746 | RNF144A | ring finger protein 144A | 0.000631 | 0.88 | 0.67 |
| 228109_at | NM_006909 | RASGRF2 | Ras protein-specific | 0.000633 | 0.84 | 0.54 |
| | | | guanine nucleotide- | | | |
| | | | releasing factor 2 | | | |
| 204099_at | NM_001003801 | SMARCD3 | SWI/SNF related, | 0.000634 | 0.94 | 1.63 |
| | | | matrix associated, actin | | | |
| | | | dependent regulator of | | | |
| | | | chromatin, subfamily d, | | | |
| | | | member 3 | | | |
| 204247_s_at | NM_004935 | CDK5 | cyclin-dependent | 0.000637 | 1.00 | 1.28 |
| | | | kinase 5 | | | |
| 218532_s_at | NM_001034850 | FAM134B | family with sequence | 0.000651 | 0.93 | 0.73 |
| | | | similarity 134, member B | | | |
| 230531_at | NM_004977 | KCNC3 | potassium voltage- | 0.000661 | 0.97 | 1.14 |
| | | | gated channel, Shaw- | | | |
| | | | related subfamily, | | | |
| | | | member 3 | | | |
| 232065_x_at | NM_033319 | CENPL | centromere protein L | 0.000661 | 1.09 | 0.82 |
| 243982_at | NM_017658 | KLHL28 | Kelch-like 28 | 0.000668 | 0.95 | 0.80 |
| | | | (Drosophila) | | | |
| 229235_at | NM_032815 | NFATC2IP | nuclear factor of | 0.000681 | 1.02 | 0.75 |
| | | | activated T-cells, | | | |
| | | | cytoplasmic, | | | |
| | | | calcineurin-dependent | | | |
| | | | 2 interacting protein | | | |
| 203429_s_at | NM_014283 | C1orf9 | chromosome 1 open | 0.000682 | 1.00 | 0.88 |
| | | | reading frame 9 | | | |
| 1559413_at | NM_152772 | TCP11L2 | t-complex 11 (mouse)- | 0.000685 | 0.88 | 0.74 |
| | | | like 2 | | | |
| 209881_s_at | NM_001014987 | LAT | linker for activation of T | 0.000691 | 0.96 | 0.77 |
| | | | cells | | | |
| 204214_s_at | NM_006834 | RAB32 | RAB32, member RAS | 0.000695 | 0.84 | 1.24 |
| | | | oncogene family | | | |
| 228359_at | NM_032873 | STS-1 | Cbl-interacting protein | 0.000696 | 0.97 | 1.43 |
| | | | Sts-1 | | | |
| 205310_at | NM_001080469 | FBXO46 | F-box protein 46 | 0.000698 | 0.91 | 0.85 |
| 227809_at | NM_198581 | ZC3H6 | zinc finger CCCH-type | 0.000698 | 0.97 | 0.81 |
| | | | containing 6 | | | |
| 210844_x_at | NM_001903 | CTNNA1 | catenin (cadherin- | 0.000699 | 0.98 | 1.22 |
| | | | associated protein), | | | |
| | | | alpha 1, 102 kDa | | | |
| 218764_at | NM_006255 | PRKCH | protein kinase C, eta | 0.000701 | 0.94 | 0.75 |
| 221918_at | NM_002595 | PCTK2 | PCTAIRE protein | 0.000703 | 0.88 | 0.84 |
| | | | kinase 2 | | | |
| 226039_at | NM_012214 | MGAT4A | mannosyl (alpha-1,3-)- | 0.000712 | 0.93 | 0.76 |
| | | | glycoprotein beta-1,4- | | | |
| | | | N- | | | |
| | | | acetylglucosaminyltransferase, | | | |
| | | | isozyme A | | | |
| 224734_at | NM_002128 | HMGB1 | high-mobility group box 1 | 0.000719 | 0.98 | 0.85 |
| 224027_at | NM_148672 | CCL28 | chemokine (C-C motif) | 0.000727 | 0.93 | 0.79 |
| | | | ligand 28 | | | |
| 234978_at | NM_152313 | SLC36A4 | solute carrier family 36 | 0.000734 | 0.91 | 1.14 |
| | | | (proton/amino acid | | | |
| | | | symporter), member 4 | | | |
| 209870_s_at | NM_005503 | APBA2 | amyloid beta (A4) | 0.000747 | 0.88 | 0.80 |
| | | | precursor protein- | | | |
| | | | binding, family A, | | | |
| | | | member 2 (X11-like) | | | |
| 229725_at | NM_001009185 | ACSL6 | Acyl-CoA synthetase | 0.000754 | 0.95 | 0.55 |
| | | | long-chain family | | | |
| | | | member 6 | | | |
| 46665_at | NM_017789 | SEMA4C | sema domain, | 0.000763 | 0.88 | 0.75 |
| | | | immunoglobulin | | | |
| | | | domain (Ig), | | | |
| | | | transmembrane | | | |
| | | | domain (TM) and short | | | |
| | | | cytoplasmic domain, | | | |
| | | | (semaphorin) 4C | | | |
| 219972_s_at | NM_022495 | C14orf135 | chromosome 14 open | 0.000763 | 0.97 | 0.78 |
| | | | reading frame 135 | | | |
| 200785_s_at | NM_002332 | LRP1 | low density lipoprotein- | 0.000764 | 1.12 | 1.35 |
| | | | related protein 1 | | | |
| | | | (alpha-2-macroglobulin | | | |
| | | | receptor) | | | |
| 204520_x_at | NM_014577 | BRD1 | bromodomain | 0.000776 | 0.97 | 0.89 |
| | | | containing 1 | | | |
| 205583_s_at | NM_001039210 | CXorf45 | chromosome X open | 0.00078 | 0.91 | 0.74 |
| | | | reading frame 45 | | | |
| 212454_x_at | NM_005463 | HNRPDL | Heterogeneous nuclear | 0.000782 | 1.06 | 0.88 |
| | | | ribonucleoprotein D- | | | |
| | | | like | | | |
| 213587_s_at | NM_001100592 | ATP6V0E2 | ATPase, H+ | 0.000788 | 0.94 | 0.78 |
| | | | transporting V0 subunit | | | |
| | | | e2 | | | |
| 225245_x_at | NM_018267 | H2AFJ | H2A histone family, | 0.00079 | 0.98 | 1.33 |
| | | | member J | | | |
| 227361_at | — | HS3ST3B1 | heparan sulfate | 0.000793 | 0.82 | 0.62 |
| | | | (glucosamine) 3-O- | | | |
| | | | sulfotransferase 3B1 | | | |
| 219724_s_at | NM_001098815 | KIAA0748 | KIAA0748 | 0.000798 | 1.00 | 0.73 |
| 204614_at | NM_002575 | SERPINB2 | serpin peptidase | 0.000801 | 1.47 | 2.17 |
| | | | inhibitor, clade B | | | |
| | | | (ovalbumin), member 2 | | | |
| 210054_at | NM_024511 | C4orf15 | chromosome 4 open | 0.000806 | 0.96 | 0.86 |
| | | | reading frame 15 | | | |
| 226100_at | NM_018682 | MLL5 | myeloid/lymphoid or | 0.000808 | 0.98 | 0.84 |
| | | | mixed-lineage | | | |
| | | | leukemia 5 (trithorax | | | |
| | | | homolog, Drosophila) | | | |
| 224842_at | NM_015092 | SMG1 | PI-3-kinase-related | 0.000809 | 0.99 | 0.92 |
| | | | kinase SMG-1 | | | |
| 228941_at | NM_001013620 | ALG10B | asparagine-linked | 0.000811 | 1.03 | 0.67 |
| | | | glycosylation 10 | | | |
| | | | homolog B (yeast, | | | |
| | | | alpha-1,2- | | | |
| | | | glucosyltransferase) | | | |
| 203723_at | NM_002221 | ITPKB | inositol 1,4,5- | 0.000812 | 0.89 | 0.83 |
| | | | trisphosphate 3-kinase B | | | |
| 222688_at | NM_018367 | PHCA | phytoceramidase, | 0.000839 | 1.20 | 1.45 |
| | | | alkaline | | | |
| 214741_at | NM_003432 | ZNF131 | zinc finger protein 131 | 0.000842 | 1.04 | 0.84 |
| 228370_at | NM_003097 | SNRPN | Small nuclear | 0.000843 | 0.82 | 0.61 |
| | | | ribonucleoprotein | | | |
| | | | polypeptide N | | | |
| 208963_x_at | NM_013402 | FADS1 | fatty acid desaturase 1 | 0.000844 | 1.42 | 1.73 |
| 221510_s_at | NM_014905 | GLS | glutaminase | 0.000845 | 1.04 | 0.77 |
| 218428_s_at | NM_001037872 | REV1 | REV1 homolog (S. cerevisiae) | 0.000846 | 1.01 | 0.89 |
| 218362_s_at | NM_014953 | DIS3 | DIS3 mitotic control | 0.00085 | 0.92 | 0.86 |
| | | | homolog (S. cerevisiae) | | | |
| 242644_at | NM_152468 | TMC8 | Transmembrane | 0.000853 | 0.94 | 0.72 |
| | | | channel-like 8 | | | |
| 49452_at | NM_001093 | ACACB | acetyl-Coenzyme A | 0.000854 | 0.88 | 0.71 |
| | | | carboxylase beta | | | |
| 209570_s_at | NM_001040101 | D4S234E | DNA segment on | 0.000859 | 1.25 | 0.80 |
| | | | chromosome 4 | | | |
| | | | (unique) 234 | | | |
| | | | expressed sequence | | | |
| 223019_at | NM_001035534 | FAM129B | family with sequence | 0.00086 | 1.09 | 1.54 |
| | | | similarity 129, member B | | | |
| 230852_at | NM_145064 | STAC3 | SH3 and cysteine rich | 0.000876 | 0.99 | 1.25 |
| | | | domain 3 | | | |
| 220485_s_at | NM_001039508 | SIRPG | signal-regulatory | 0.000878 | 0.96 | 0.75 |
| | | | protein gamma | | | |
| 221011_s_at | NM_030915 | LBH | limb bud and heart | 0.000878 | 0.93 | 0.66 |
| | | | development homolog | | | |
| | | | (mouse) | | | |
| 222876_s_at | NM_018404 | CENTA2 | centaurin, alpha 2 | 0.000879 | 1.19 | 1.60 |
| 231853_at | NM_016261 | TUBD1 | tubulin, delta 1 | 0.000879 | 0.95 | 0.81 |
| 219038_at | NM_001085354 | MORC4 | MORC family CW-type | 0.000882 | 0.96 | 0.78 |
| | | | zinc finger 4 | | | |
| 201231_s_at | NM_001428 | ENO1 | enolase 1, (alpha) | 0.000885 | 1.05 | 1.19 |
| 209504_s_at | NM_021200 | PLEKHB1 | pleckstrin homology | 0.000886 | 0.89 | 0.71 |
| | | | domain containing, | | | |
| | | | family B (evectins) | | | |
| | | | member 1 | | | |
| 215245_x_at | NM_002024 | FMR1 | fragile X mental | 0.000888 | 0.94 | 0.84 |
| | | | retardation 1 | | | |
| 47571_at | NM_007345 | ZNF236 | zinc finger protein 236 | 0.000888 | 1.06 | 0.94 |
| 205288_at | NM_003672 | CDC14A | CDC14 cell division | 0.000894 | 1.08 | 0.77 |
| | | | cycle 14 homolog A (S. cerevisiae) | | | |
| 218552_at | NM_018281 | ECHDC2 | enoyl Coenzyme A | 0.000894 | 1.02 | 0.76 |
| | | | hydratase domain | | | |
| | | | containing 2 | | | |
| 209149_s_at | NM_001014842 | TM9SF1 | transmembrane 9 | 0.000894 | 0.74 | 1.10 |
| | | | superfamily member 1 | | | |
| 216713_at | NM_001013406 | KRIT1 | KRIT1, ankyrin repeat | 0.000899 | 1.10 | 0.84 |
| | | | containing | | | |
| 1569652_at | NM_004529 | MLLT3 | myeloid/lymphoid or | 0.000903 | 0.87 | 0.59 |
| | | | mixed-lineage | | | |
| | | | leukemia (trithorax | | | |
| | | | homolog, Drosophila); | | | |
| | | | translocated to, 3 | | | |
| 218422_s_at | NM_022118 | RBM26 | RNA binding motif | 0.000906 | 1.02 | 0.83 |
| | | | protein 26 | | | |
| 234923_at | NM_014990 | GARNL1 | GTPase activating | 0.000907 | 0.94 | 0.75 |
| | | | Rap/RanGAP domain- | | | |
| | | | like 1 | | | |
| 222141_at | NM_032775 | KLHL22 | kelch-like 22 | 0.000907 | 1.02 | 0.83 |
| | | | (Drosophila) | | | |
| 207283_at | NR_002229 | RPL23AP13 | ribosomal protein L23a | 0.000917 | 1.05 | 0.81 |
| | | | pseudogene 13 | | | |
| 205211_s_at | NM_004292 | RIN1 | Ras and Rab interactor 1 | 0.00092 | 0.96 | 1.18 |
| 203665_at | NM_002133 | HMOX1 | heme oxygenase | 0.000921 | 1.09 | 1.44 |
| | | | (decycling) 1 | | | |
| 226465_s_at | NM_032195 | SON | SON DNA binding | 0.000932 | 1.00 | 0.85 |
| | | | protein | | | |
| 228594_at | NM_001085411 | C5orf33 | chromosome 5 open | 0.000937 | 1.01 | 0.86 |
| | | | reading frame 33 | | | |
| 211339_s_at | NM_005546 | ITK | IL2-inducible T-cell | 0.000942 | 1.00 | 0.71 |
| | | | kinase | | | |
| AFFX- | NM_002046 | GAPDH | glyceraldehyde-3- | 0.000944 | 0.97 | 1.17 |
| HUMGAPDH/ | | | phosphate | | | |
| M33197_5_at | | | dehydrogenase | | | |
| 205590_at | NM_005739 | RASGRP1 | RAS guanyl releasing | 0.000946 | 0.93 | 0.69 |
| | | | protein 1 (calcium and | | | |
| | | | DAG-regulated) | | | |
| 224439_x_at | NM_014245 | RNF7 | ring finger protein 7 | 0.000946 | 1.06 | 1.16 |
| 219441_s_at | NM_024652 | LRRK1 | leucine-rich repeat | 0.000954 | 0.81 | 1.11 |
| | | | kinase 1 | | | |
| 1553165_at | NM_007247 | AP1GBP1 | AP1 gamma subunit | 0.000954 | 1.00 | 0.82 |
| | | | binding protein 1 | | | |
| 225942_at | NM_020726 | NLN | neurolysin | 0.000956 | 1.29 | 1.22 |
| | | | (metallopeptidase M3 | | | |
| | | | family) | | | |
| 216202_s_at | NM_004863 | SPTLC2 | serine | 0.000959 | 0.76 | 1.37 |
| | | | palmitoyltransferase, | | | |
| | | | long chain base | | | |
| | | | subunit 2 | | | |
| 209218_at | NM_003129 | SQLE | squalene epoxidase | 0.000964 | 1.37 | 1.78 |
| 206965_at | NM_007249 | KLF12 | Kruppel-like factor 12 | 0.000964 | 0.83 | 0.56 |
| 218911_at | NM_006530 | YEATS4 | YEATS domain | 0.000976 | 0.97 | 0.77 |
| | | | containing 4 | | | |
| 228680_at | NM_007054 | KIF3A | kinesin family member | 0.000977 | 0.96 | 0.73 |
| | | | 3A | | | |
| 202258_s_at | NM_014887 | PFAAP5 | phosphonoformate | 0.000979 | 1.06 | 0.94 |
| | | | immuno-associated | | | |
| | | | protein 5 | | | |
| 201486_at | NM_002902 | RCN2 | reticulocalbin 2, EF- | 0.000983 | 1.07 | 0.84 |
| | | | hand calcium binding | | | |
| | | | domain | | | |
| 241871_at | NM_001744 | CAMK4 | calcium/calmodulin- | 0.000985 | 0.90 | 0.53 |
| | | | dependent protein | | | |
| | | | kinase IV | | | |
| 212633_at | NM_015323 | KIAA0776 | KIAA0776 | 0.000987 | 1.14 | 0.90 |
| 218885_s_at | NM_024642 | GALNT12 | UDP-N-acetyl-alpha-D- | 0.000989 | 0.85 | 0.70 |
| | | | galactosamine:polypeptide | | | |
| | | | N- | | | |
| | | | acetylgalactosaminyltransferase | | | |
| | | | 12 (GalNAc- | | | |
| | | | T12) | | | |
| 212400_at | NM_001035254 | FAM102A | family with sequence | 0.00099 | 0.93 | 0.67 |
| | | | similarity 102, member A | | | |
| 222744_s_at | NM_018196 | TMLHE | trimethyllysine | 0.000991 | 0.92 | 1.21 |
| | | | hydroxylase, epsilon | | | |
| 206542_s_at | NM_003070 | SMARCA2 | SWI/SNF related, | 0.000993 | 0.98 | 0.86 |
| | | | matrix associated, actin | | | |
| | | | dependent regulator of | | | |
| | | | chromatin, subfamily a, | | | |
| | | | member 2 | | | |
| 202617_s_at | NM_001110792 | MECP2 | methyl CpG binding | 0.000996 | 0.92 | 0.87 |
| | | | protein 2 (Rett | | | |
| | | | syndrome) | | | |
| 1560703_at | NM_000625 | NOS2A | Nitric oxide synthase | 0.001 | 0.89 | 0.72 |
| | | | 2A (inducible, | | | |
| | | | hepatocytes) | | | |

TABLE 3
Survival Genes - Note that genes are listed in order of decreasing
statistical significance/preference.

| | | Gene | | Logrank | |
|---|---|---|---|---|---|
| Probe set | RefSeq | symbol | Gene Description | test, p-value | Coefficient |
| 218532_s_at | NM_001034850 | FAM134B | family with sequence | 0.0000188 | 0.191890372 |
| | | | similarity 134, member B | | |
| 226039_at | NM_012214 | MGAT4A | mannosyl (alpha-1,3-)- | 0.0000645 | 0.206097886 |
| | | | glycoprotein beta-1,4- | | |
| | | | N- | | |
| | | | acetylglucosaminyltransferase, | | |
| | | | isozyme A | | |
| 212655_at | NM_015144 | ZCCHC14 | zinc finger, CCHC | 0.0000688 | 0.439999867 |
| | | | domain containing 14 | | |
| 206545_at | NM_006139 | CD28 | CD28 molecule | 0.0000863 | 0.451195806 |
| 203939_at | NM_002526 | NT5E | 5′-nucleotidase, ecto | 0.0000945 | 0.138687171 |
| | | | (CD73) | | |
| 228109_at | NM_006909 | RASGRF2 | Ras protein-specific | 0.0001183 | 0.251103243 |
| | | | guanine nucleotide- | | |
| | | | releasing factor 2 | | |
| 1553132_a_at | NM_152332 | TC2N | tandem C2 domains, | 0.0001366 | 0.3599156 |
| | | | nuclear | | |
| 201656_at | NM_000210 | ITGA6 | integrin, alpha 6 | 0.0001632 | 0.264869311 |
| 214582_at | NM_000922 | PDE3B | phosphodiesterase 3B, | 0.0002216 | 0.300332089 |
| | | | cGMP-inhibited | | |
| 201677_at | NM_001006109 | C3orf37 | Chromosome 3 open | 0.0002250 | 0.082398123 |
| | | | reading frame 37 | | |
| 203408_s_at | NM_002971 | SATB1 | SATB homeobox 1 | 0.0003166 | 0.105602874 |
| 225864_at | NM_174911 | FAM84B | family with sequence | 0.0003358 | 0.386161889 |
| | | | similarity 84, member B | | |
| 209674_at | NM_004075 | CRY1 | cryptochrome 1 | 0.0003426 | 0.386134127 |
| | | | (photolyase-like) | | |
| 209657_s_at | NM_004506 | HSF2 | heat shock | 0.0003643 | 0.154506655 |
| | | | transcription factor 2 | | |
| 223092_at | NM_054027 | ANKH | ankylosis, progressive | 0.0004883 | 0.264908361 |
| | | | homolog (mouse) | | |
| 205259_at | NM_000901 | NR3C2 | nuclear receptor | 0.0005284 | 0.305743767 |
| | | | subfamily 3, group C, | | |
| | | | member 2 | | |
| 205005_s_at | NM_004808 | NMT2 | N-myristoyltransferase 2 | 0.0005364 | 0.465298698 |
| 211339_s_at | NM_005546 | ITK | IL2-inducible T-cell | 0.0005474 | 0.328170825 |
| | | | kinase | | |
| 218473_s_at | NM_024656 | GLT25D1 | glycosyltransferase 25 | 0.0005685 | 25.88043253 |
| | | | domain containing 1 | | |
| 1559097_at | — | C14orf64 | chromosome 14 open | 0.0005902 | 0.268401623 |
| | | | reading frame 64 | | |
| 220485_s_at | NM_001039508 | SIRPG | signal-regulatory | 0.0006799 | 0.197558497 |
| | | | protein gamma | | |
| 209881_s_at | NM_001014987 | LAT | linker for activation of T | 0.0008250 | 0.164825676 |
| | | | cells | | |
| 224968_at | NM_080667 | CCDC104 | coiled-coil domain | 0.0008433 | 0.310483663 |
| | | | containing 104 | | |
| 227361_at | — | HS3ST3B1 | heparan sulfate | 0.0009171 | 0.4056675 |
| | | | (glucosamine) 3-O- | | |
| | | | sulfotransferase 3B1 | | |
| 209870_s_at | NM_005503 | APBA2 | amyloid beta (A4) | 0.0009945 | 0.143757862 |
| | | | precursor protein- | | |
| | | | binding, family A, | | |
| | | | member 2 (X11-like) | | |
| 46665_at | NM_017789 | SEMA4C | sema domain, | 0.0010157 | 0.175513963 |
| | | | immunoglobulin | | |
| | | | domain (Ig), | | |
| | | | transmembrane | | |
| | | | domain (TM) and short | | |
| | | | cytoplasmic domain, | | |
| | | | (semaphorin) 4C | | |
| 205288_at | NM_003672 | CDC14A | CDC14 cell division | 0.0012955 | 0.20206578 |
| | | | cycle 14 homolog A (S. cerevisiae) | | |
| 230078_at | NM_016340 | RAPGEF6 | Rap guanine | 0.0013072 | 0.12377883 |
| | | | nucleotide exchange | | |
| | | | factor (GEF) 6 | | |
| 235048_at | NM_015566 | KIAA0888 | KIAA0888 protein | 0.0013164 | 0.418246256 |
| 214049_x_at | NM_006137 | CD7 | CD7 molecule | 0.0014992 | 0.217794866 |
| 219387_at | NM_018084 | CCDC88A | coiled-coil domain | 0.0016015 | 2.4251108 |
| | | | containing 88A | | |
| 212538_at | NM_015296 | DOCK9 | dedicator of cytokinesis 9 | 0.0016303 | 0.3086959 |
| 204040_at | NM_014746 | RNF144A | ring finger protein 144A | 0.0016546 | 0.386430727 |
| 211282_x_at | NM_001039664 | TNFRSF25 | tumor necrosis factor | 0.0017567 | 0.461061973 |
| | | | receptor superfamily, | | |
| | | | member 25 | | |
| 222895_s_at | NM_022898 | BCL11B | B-cell CLL/lymphoma | 0.0019714 | 0.442108802 |
| | | | 11B (zinc finger | | |
| | | | protein) | | |
| 226247_at | NM_001001974 | PLEKHA1 | pleckstrin homology | 0.0021405 | 0.360753139 |
| | | | domain containing, | | |
| | | | family A | | |
| | | | (phosphoinositide | | |
| | | | binding specific) | | |
| | | | member 1 | | |
| 229725_at | NM_001009185 | ACSL6 | Acyl-CoA synthetase | 0.0027287 | 0.334884067 |
| | | | long-chain family | | |
| | | | member 6 | | |
| 1556402_at | — | FLJ46446 | Hypothetical gene | 0.0030901 | 0.327796466 |
| | | | supported by | | |
| | | | AK128305 | | |
| 241871_at | NM_001744 | CAMK4 | calcium/calmodulin- | 0.0032142 | 0.424186978 |
| | | | dependent protein | | |
| | | | kinase IV | | |
| 210031_at | NM_000734 | CD247 | CD247 molecule | 0.0032530 | 0.400517646 |
| 214195_at | NM_000391 | TPP1 | tripeptidyl peptidase I | 0.0036692 | 284.1586694 |
| 212981_s_at | NM_014719 | FAM115A | family with sequence | 0.0039653 | 0.2380392 |
| | | | similarity 115, member A | | |
| 202664_at | NM_001077269 | WIPF1 | WAS/WASL interacting | 0.0040573 | 0.032530289 |
| | | | protein family, member 1 | | |
| 205434_s_at | NM_001012987 | AAK1 | AP2 associated kinase 1 | 0.0042792 | 0.334485861 |
| 226682_at | — | LOC283666 | hypothetical protein | 0.0045761 | 0.507380631 |
| | | | LOC283666 | | |
| 212609_s_at | NM_005465 | AKT3 | V-akt murine thymoma | 0.0047050 | 0.25152629 |
| | | | viral oncogene | | |
| | | | homolog 3 (protein | | |
| | | | kinase B, gamma) | | |
| 209570_s_at | NM_001040101 | D4S234E | DNA segment on | 0.0047217 | 0.418667259 |
| | | | chromosome 4 | | |
| | | | (unique) 234 | | |
| | | | expressed sequence | | |
| 202970_at | NM_003583 | DYRK2 | dual-specificity | 0.0049630 | 0.16334198 |
| | | | tyrosine-(Y)- | | |
| | | | phosphorylation | | |
| | | | regulated kinase 2 | | |
| 214470_at | NM_002258 | KLRB1 | killer cell lectin-like | 0.0049710 | 0.525559684 |
| | | | receptor subfamily B, | | |
| | | | member 1 | | |
| 202704_at | NM_005749 | TOB1 | transducer of ERBB2, 1 | 0.0051841 | 0.162338576 |
| 212259_s_at | NM_020524 | PBXIP1 | pre-B-cell leukemia | 0.0057089 | 0.158782145 |
| | | | homeobox interacting | | |
| | | | protein 1 | | |
| 214442_s_at | NM_004671 | PIAS2 | protein inhibitor of | 0.0057920 | 2.837870356 |
| | | | activated STAT, 2 | | |
| 218764_at | NM_006255 | PRKCH | protein kinase C, eta | 0.0062209 | 0.345585393 |
| 209604_s_at | NM_001002295 | GATA3 | GATA binding protein 3 | 0.0075146 | 0.437278076 |
| 205590_at | NM_005739 | RASGRP1 | RAS guanyl releasing | 0.0087734 | 0.426211611 |
| | | | protein 1 (calcium and | | |
| | | | DAG-regulated) | | |
| 209884_s_at | NM_003615 | SLC4A7 | solute carrier family 4, | 0.0090340 | 0.218893307 |
| | | | sodium bicarbonate | | |
| | | | cotransporter, member 7 | | |
| 210054_at | NM_024511 | C4orf15 | chromosome 4 open | 0.0100695 | 0.131111638 |
| | | | reading frame 15 | | |
| 204642_at | NM_001400 | EDG1 | endothelial | 0.0103863 | 0.379777976 |
| | | | differentiation, | | |
| | | | sphingolipid G-protein- | | |
| | | | coupled receptor, 1 | | |
| 227626_at | NM_133367 | PAQR8 | progestin and adipoQ | 0.0104034 | 0.236527598 |
| | | | receptor family | | |
| | | | member VIII | | |
| 218723_s_at | NM_014059 | C13orf15 | chromosome 13 open | 0.0107424 | 0.458785487 |
| | | | reading frame 15 | | |
| 47560_at | NM_001008701 | LPHN1 | latrophilin 1 | 0.0110522 | 0.321941043 |
| 218885_s_at | NM_024642 | GALNT12 | UDP-N-acetyl-alpha-D- | 0.0118176 | 0.093592166 |
| | | | galactosamine:polypeptide | | |
| | | | N- | | |
| | | | acetylgalactosaminyltransferase | | |
| | | | 12 (GalNAc- | | |
| | | | T12) | | |
| 225619_at | NM_001040153 | SLAIN1 | SLAIN motif family, | 0.0120944 | 0.411766455 |
| | | | member 1 | | |
| 209368_at | NM_001979 | EPHX2 | epoxide hydrolase 2, | 0.0121956 | 0.16566281 |
| | | | cytoplasmic | | |
| 209798_at | NM_002519 | NPAT | nuclear protein, ataxiatelangiectasia | 0.0123399 | 0.21571892 |
| | | | locus | | |
| 218454_at | NM_024829 | FLJ22662 | hypothetical protein | 0.0125517 | 6.434077987 |
| | | | FLJ22662 | | |
| 228065_at | NM_182557 | BCL9L | B-cell CLL/lymphoma | 0.0128291 | 0.239389548 |
| | | | 9-like | | |
| 203665_at | NM_002133 | HMOX1 | heme oxygenase | 0.0133210 | 3.640692548 |
| | | | (decycling) 1 | | |
| 243492_at | NM_053055 | THEM4 | Thioesterase | 0.0134817 | 0.319867508 |
| | | | superfamily member 4 | | |
| 224833_at | NM_005238 | ETS1 | v-ets erythroblastosis | 0.0141079 | 0.365618531 |
| | | | virus E26 oncogene | | |
| | | | homolog 1 (avian) | | |
| 213908_at | NR_003521 | WHDC1L1 | WAS protein homology | 0.0143315 | 0.261281401 |
| | | | region 2 domain | | |
| | | | containing 1-like 1 | | |
| 228950_s_at | NM_001002292 | GPR177 | G protein-coupled | 0.0144013 | 0.557215355 |
| | | | receptor 177 | | |
| 204773_at | NM_004512 | IL11RA | interleukin 11 receptor, | 0.0162697 | 0.407147756 |
| | | | alpha | | |
| 204099_at | NM_001003801 | SMARCD3 | SWI/SNF related, | 0.0165894 | 2.41590844 |
| | | | matrix associated, actin | | |
| | | | dependent regulator of | | |
| | | | chromatin, subfamily d, | | |
| | | | member 3 | | |
| 212400_at | NM_001035254 | FAM102A | family with sequence | 0.0185661 | 0.403641178 |
| | | | similarity 102, member A | | |
| 205603_s_at | NM_006729 | DIAPH2 | diaphanous homolog 2 | 0.0193058 | 4.169717361 |
| | | | (Drosophila) | | |
| 228853_at | XM_001125680 | LOC730432 | similar to | 0.0200269 | 0.150319437 |
| | | | serine/threonine/tyrosine | | |
| | | | interacting protein | | |
| 237033_at | NM_001042693 | MGC52498 | hypothetical protein | 0.0200330 | 0.351911723 |
| | | | MGC52498 | | |
| 221918_at | NM_002595 | PCTK2 | PCTAIRE protein | 0.0201972 | 0.095861991 |
| | | | kinase 2 | | |
| 212675_s_at | NM_015147 | CEP68 | centrosomal protein | 0.0203975 | 0.335424361 |
| | | | 68 kDa | | |
| 235125_x_at | NM_198549 | FAM73A | family with sequence | 0.0209776 | 0.279735767 |
| | | | similarity 73, member A | | |
| 206761_at | NM_005816 | CD96 | CD96 molecule | 0.0213812 | 0.549088648 |
| 200965_s_at | NM_001003407 | ABLIM1 | actin binding LIM | 0.0225864 | 0.451561647 |
| | | | protein 1 | | |
| 231124_x_at | NM_001033667 | LY9 | lymphocyte antigen 9 | 0.0243982 | 0.506529706 |
| 202419_at | NM_002035 | FVT1 | follicular lymphoma | 0.0245457 | 0.249130374 |
| | | | variant translocation 1 | | |
| 205936_s_at | NM_002115 | HK3 | hexokinase 3 (white | 0.0254338 | 2.921378857 |
| | | | cell) | | |
| 230490_x_at | NM_012425 | RSU1 | Ras suppressor protein 1 | 0.0258599 | 0.14389267 |
| 209504_s_at | NM_021200 | PLEKHB1 | pleckstrin homology | 0.0259312 | 0.39679049 |
| | | | domain containing, | | |
| | | | family B (evectins) | | |
| | | | member 1 | | |
| 49452_at | NM_001093 | ACACB | acetyl-Coenzyme A | 0.0262238 | 0.18297323 |
| | | | carboxylase beta | | |
| 201560_at | NM_013943 | CLIC4 | chloride intracellular | 0.0262927 | 3.251909183 |
| | | | channel 4 | | |
| 215245_x_at | NM_002024 | FMR1 | fragile X mental | 0.0263504 | 0.070926355 |
| | | | retardation 1 | | |
| 221221_s_at | NM_017415 | KLHL3 | kelch-like 3 | 0.0278018 | 0.483951344 |
| | | | (Drosophila) | | |
| 224027_at | NM_148672 | CCL28 | chemokine (C-C motif) | 0.0283153 | 0.116254904 |
| | | | ligand 28 | | |
| 222585_x_at | NM_016618 | KRCC1 | lysine-rich coiled-coil 1 | 0.0287697 | 13.76693584 |
| 222435_s_at | NM_016021 | UBE2J1 | ubiquitin-conjugating | 0.0295370 | 3.05946191 |
| | | | enzyme E2, J1 (UBC6 | | |
| | | | homolog, yeast) | | |
| 228423_at | NM_001039580 | MAP9 | microtubule-associated | 0.0308351 | 0.490096457 |
| | | | protein 9 | | |
| 227639_at | NM_005482 | PIGK | phosphatidylinositol | 0.0312002 | 0.244295712 |
| | | | glycan anchor | | |
| | | | biosynthesis, class K | | |
| 208807_s_at | NM_001005271 | CHD3 | chromodomain | 0.0312319 | 0.187691851 |
| | | | helicase DNA binding | | |
| | | | protein 3 | | |
| 218911_at | NM_006530 | YEATS4 | YEATS domain | 0.0319970 | 0.28642978 |
| | | | containing 4 | | |
| 224698_at | NM_020728 | FAM62B | family with sequence | 0.0324392 | 0.341961555 |
| | | | similarity 62 (C2 | | |
| | | | domain containing) | | |
| | | | member B | | |
| 1552426_a_at | NM_025141 | TM2D3 | TM2 domain containing 3 | 0.0348942 | 0.063258104 |
| 222820_at | NM_018996 | TNRC6C | trinucleotide repeat | 0.0372469 | 0.348442308 |
| | | | containing 6C | | |
| 211336_x_at | NM_001081637 | LILRB1 | leukocyte | 0.0418491 | 4.199248492 |
| | | | immunoglobulin-like | | |
| | | | receptor, subfamily B | | |
| | | | (with TM and ITIM | | |
| | | | domains), member 1 | | |
| 201486_at | NM_002902 | RCN2 | reticulocalbin 2, EF- | 0.0466717 | 0.235273316 |
| | | | hand calcium binding | | |
| | | | domain | | |
| 227900_at | NM_170662 | CBLB | Cas-Br-M (murine) | 0.0467392 | 0.586176881 |
| | | | ecotropic retroviral | | |
| | | | transforming sequence b | | |
| 204484_at | NM_002646 | PIK3C2B | phosphoinositide-3- | 0.0467784 | 0.450595829 |
| | | | kinase, class 2, beta | | |
| | | | polypeptide | | |
| 234978_at | NM_152313 | SLC36A4 | solute carrier family 36 | 0.0468999 | 4.702439659 |
| | | | (proton/amino acid | | |
| | | | symporter), member 4 | | |
| 218499_at | NM_001042452 | RP6- | serine/threonine | 0.0475275 | 0.14086787 |
| | | 213H19.1 | protein kinase MST4 | | |
| 1569652_at | NM_004529 | MLLT3 | myeloid/lymphoid or | 0.0484774 | 0.517632562 |
| | | | mixed-lineage | | |
| | | | leukemia (trithorax | | |
| | | | homolog, Drosophila); | | |
| | | | translocated to, 3 | | |
| 210102_at | NM_014622 | LOH11CR2A | loss of heterozygosity, | 0.0498673 | 2.706868224 |
| | | | 11, chromosomal | | |
| | | | region 2, gene A | | |

TABLE 4

| Gene Symbol | ANOVA p | NYHA I-II/Ctrl | NYHA III-IV/Ctrl |
|---|---|---|---|
| CALM1 | 0.000105 | 0.98 | 0.84 |
| PIK3C2B | 0.000148 | 0.87 | 0.63 |
| CD28 | 0.000321 | 1.01 | 0.58 |
| CD247 | 0.000573 | 0.88 | 0.68 |
| LAT | 0.000691 | 0.96 | 0.77 |
| ITK | 0.000942 | 1.00 | 0.71 |
| RASGRP1 | 0.000946 | 0.93 | 0.69 |
| CAMK4 | 0.000985 | 0.90 | 0.53 |

### REFERENCES

Braunwald, E. 2008. Biomarkers in heart failure. N Engl J Med 358:2148-2159
Cunha-Neto, E., V. J. Dzau, P. D. Allen, D. Stamatiou, L. Benvenutti, M. L. Higuchi, N. S. Koyama, J. S. Silva, J. Kalil, and C. C. Liew. 2005. Cardiac gene expression profiling provides evidence for cytokinopathy as a molecular mechanism in Chagas' disease cardiomyopathy. Am J. Pathol. 167:305-313.
Kittleson, M. M., K. M. Minhas, R. A. Irizarry, S. Q. Ye, G. Edness, E. Breton, J. V. Conte, G. Tomaselli, J. G. Garcia, and J. M. Hare. 2005. Gene expression analysis of ischemic and nonischemic cardiomyopathy: shared and distinct genes in the development of heart failure. Physiol Genomics 21:299-307
Ma, J. and C. C. Liew. 2003. Gene profiling identifies secreted protein transcripts from peripheral blood cells in coronary artery disease. J. Mol. Cell Cardiol. 35:993-998
Ma, J., A. A. Dempsey, D. Stamatiou, K. W. Marshall, and C. C. Liew. Identifying leukocyte gene expression patterns associated with plasma lipid levels in human subjects. Atherosclerosis. 2007, 191(1):63-72
Mann, D. L. and M. R. Bristow. 2005. Mechanisms and models in heart failure: the biomechanical model and beyond. Circulation 111:2837-2849
Roger, V. L., S. A. Weston, M. M. Redfield, J. P. Hellermann-Homan, J. Killian, B. P. Yawn, and S. J. Jacobsen. 2004. Trends in heart failure incidence and survival in a community-based population. JAMA 292:344-350
Rosamond, W., K. Flegal, G. Friday, et al. American Heart Association Statistics Committee and Stroke Statistics Subcommittee. Heart disease and stroke statistics—2007 update: a report from the American Heart Association Statistics Committee and Stroke Statistics Subcommittee. Circulation. 2007, 115: e69-171
Schocken, D. D., E. J. Benjamin, G. C. Fonarow, H. M. Krumholz, D. Levy, G. A. Mensah, J. Narula, E. S. Shor, J. B. Young, and Y. Hong. 2008. Prevention of heart failure: a scientific statement from the American Heart Association Councils on Epidemiology and Prevention, Clinical Cardiology, Cardiovascular Nursing, and High Blood Pressure Research; Quality of Care and Outcomes Research Interdisciplinary Working Group; and Functional Genomics and Translational Biology Interdisciplinary Working Group. Circulation 117:2544-2565
Storey, J. D. and R. Tibshirani. 2003. Statistical significance for genomewide studies. Proc. Natl. Acad. Sci. U.S.A 100:9440-9445

1. A method of determining a severity of heart failure in a human test subject, the method comprising, for each gene of a set of one or more genes listed in Table 2:
a) providing test data representing a level of RNA encoded by the gene in blood of the test subject;
b) providing positive control data representing levels of RNA encoded by the gene in blood of human control subjects having a categorized severity of heart failure; and
c) comparing the level of step a) to the levels in blood of control subjects to thereby determine a value indicating whether the test data corresponds to the positive control data;
wherein a correspondence between the test data and the positive control data indicates that the test subject has the categorized severity of heart failure.

2. The method of claim 1, wherein the set of genes consists of an ASGR2 gene and a STAB1 gene.

3. The method of claim 1, wherein the categorized severity is compensated heart failure or decompensated heart failure.

4. The method of claim 1, wherein the level of RNA encoded by the gene in blood of the test subject and the levels in blood of positive control subjects are relative to a level of RNA encoded by the gene in blood of healthy test subjects.

5. The method of claim 1, further comprising determining a level of RNA encoded by the gene in blood of the test subject, thereby providing the test data.

6. The method of claim 5, further comprising determining levels of RNA encoded by the gene in blood of human subjects having the categorized severity of heart failure, thereby providing the positive control data.

7. The method of claim 1, wherein step c) is effected by:
inputting, to a computer, the test data, wherein the computer is for comparing data representing a level of RNA encoded by the gene in blood of a human subject to levels of RNA encoded by the gene in subjects having the categorized severity of heart failure, to thereby output a value indicating whether the test data corresponds to the positive control data; and
causing the computer to compare the test data to the positive control data, to thereby output the value indicating whether the test data corresponds to the positive control data.

8. A kit comprising packaging and containing, for each gene of a set of one or more of the genes listed in Table 2, a primer set capable of generating an amplification product of DNA complementary to RNA encoded, in a human subject, only by the gene.

9. The kit of claim 8, wherein the set of genes consists of an ASGR2 gene and a STAB1 gene.

10. The kit of claim 8, further comprising for a control gene, a primer set capable of generating an amplification product of DNA complementary to RNA encoded, in a human subject, only by the gene.

11. The kit of claim 8, further comprising a thermostable polymerase, a reverse transcriptase, deoxynucleotide triphosphates, nucleotide triphosphates and/or enzyme buffer.

12. The kit of claim 8, further comprising at least one labeled probe capable of selectively hybridizing to either a sense or an antisense strand of the amplification product.

13. The kit of claim 8, further comprising a computer-readable medium having instructions stored thereon that are operable when executed by a computer for comparing test data representing a level of RNA encoded by the gene in blood of a human test subject to positive control data representing levels of RNA encoded by the gene in blood of human control subjects having a categorized severity of heart failure, to thereby output data representing a value indicating whether the test data and the positive control data correspond to each other, wherein correspondence between the test data and the positive control data indicates that the test subject has the categorized severity of heart failure.

14. An isolated composition comprising, a blood sample from a test subject and for each gene of a set of one or more genes selected from the genes listed in Table 2, one or more components selected from the group consisting of exogenous RNA encoded by the gene, cDNA complementary to the RNA, an oligonucleotide which specifically hybridizes to the cDNA or the RNA under stringent conditions, a primer set capable of generating an amplification product of the cDNA complementary to RNA, and an amplification product of the cDNA.

15. The isolated composition of claim 14, wherein the set of genes consists of an ASGR2 gene and a STAB1 gene.

16. An isolated composition comprising, for each gene of a set of genes selected from the genes listed in Table 2, one or more components selected from the group consisting of: an exogenous isolated RNA encoded by the gene, cDNA complementary to the RNA, an oligonucleotide which specifically hybridizes to the cDNA or the RNA under stringent conditions, a primer set capable of generating an amplification product of the cDNA complementary to RNA, and an amplification product of the cDNA.

17. The isolated composition of claim 16, wherein the set of genes consists of an ASGR2 gene and a STAB1 gene.

18. A primer set comprising a first primer and a second primer, wherein the first primer is one of a set of primers capable of generating an amplification product of cDNA complementary to RNA encoded by a first gene, wherein the second primer is capable of generating an amplification product of cDNA complementary to RNA encoded by a second gene, and wherein the first gene and the second gene are different genes selected from the genes listed in Table 2, or composition thereof.

19. The primer set of claim 18, wherein the first gene is an ASGR2 gene, and the second gene is a STAB1 gene.
